SEQUENCE LISTING (2 sequences)

SEQ ID NO: 1 - DNA, 32 bp, Simian Virus 40
tacttatcctgtcccttttttttccacaggtg

SEQ ID NO: 2 - DNA, 43 bp, Simian Virus 40
tactaactgatatctcttctttttttttttccggaaaacaggc

(19) United States
(12) Patent Application Publication
(10) Pub. No.: US 2022/0213469 A1
(43) Pub. Date: Jul. 7, 2022

(54) METHODS AND COMPOSITIONS FOR BARCODING NUCLEIC ACID LIBRARIES AND CELL POPULATIONS

(71) Applicants: THE BROAD INSTITUTE, INC., Cambridge, MA (US); MASSACHUSETTS INSTITUTE OF TECHNOLOGY, Cambridge, MA (US)

(72) Inventors: Paul Blainey, Cambridge, MA (US); Jacob Borrajo, Cambridge, MA (US)

(21) Appl. No.: 17/607,615

(22) PCT Filed: Apr. 30, 2020
(86) PCT No.: PCT/US2020/030821

§ 371 Date: Oct. 29, 2021

Related U.S. Application Data
(60) Provisional application No. 62/840,993, filed on Apr. 30, 2019.

(51) Int. Cl. C12N 15/10; C12N 15/113; C12N 15/86

(52) U.S. Cl. CPC C12N 15/1065; C12N 15/113; C12N 15/86; C12N 2840/445; C12N 2310/11; C12N 2830/008; C12N 2740/15043; C12N 2310/1241; C12N 2830/36

(57) ABSTRACT

Method of generating a barcoded library, comprising delivering a polynucleotide into a cell, each polynucleotide comprising: (i) a sequence encoding a barcoding construct operably linked to a first promoter that is an antisense promoter, wherein the barcoding construct comprises a trans-splicing element and a barcode sequence; and a sequence encoding a perturbation element operably linked to a second promoter; generating RNA transcripts of the polynucleotide delivered into the cell, wherein the RNA transcripts comprise the barcoding construct and the perturbation element; and splicing the barcoding sequence onto endogenous RNA molecules in the cell, thereby generating a barcoded library, each member of the barcoded library comprising the barcode sequence and the endogenous RNA molecule attached with the barcode sequence.

### CROSS-REFERENCE TO RELATED APPLICATIONS

This application claims the benefit of U.S. Provisional Application No. 62/840,993, filed Apr. 30, 2019. The entire contents of the above-identified application are hereby fully incorporated herein by reference.

### STATEMENT REGARDING FEDERALLY SPONSORED RESEARCH

This invention was made with government support under Grant No. HL141005 awarded by the National Institutes of Health. The government has certain rights in the invention.

### REFERENCE TO AN ELECTRONIC SEQUENCE LISTING

The contents of the electronic sequence listing (“BROD-4030WP_ST25.txt;” Size is 686 bytes and it was created on Apr. 14, 2020) is herein incorporated by reference in its entirety.

### TECHNICAL FIELD

The subject matter disclosed herein is generally directed to methods for barcoding nucleic acid libraries and cell populations.

### BACKGROUND

Transcriptome profiling is an important method for functional characterization of cells and tissues and for obtaining information for diagnosing and treating diseases. Current methods often involve generating RNA libraries in compartmentalized wells or droplets, which limit the throughput, and can be expensive and labor-intensive. Methods that allow for generating libraries in multiple types of cell populations in a single volume are needed for increasing the throughput of transcriptome profiling assays.

### SUMMARY

In one aspect, the present disclosure provides a nucleic acid construct comprising a nucleic acid sequence encoding a barcoding construct operably linked to a first promoter that is an antisense promoter and comprises a trans-splicing element and a barcode sequence, and a nucleic acid sequence encoding one or more perturbation elements operably linked to a second promoter.

In some embodiments, the nucleic acid construct further comprises a nucleic acid sequence encoding a transcription terminator. In some embodiments, the transcription terminator is an antisense terminator. In some embodiments, the antisense promoter does not comprise a splice donor site. In some embodiments, the nucleic acid further comprises a reverse transcription primer binding site. In some embodiments, the trans-splicing element comprises a branch point, a polypyrimidine tract, a splice acceptor sequence, or a combination thereof. In some embodiments, the trans-splicing element is a ribozyme. In some embodiments, the nucleic acid construct further comprises a CRISPR-Cas guide RNA binding site. In some embodiments, the CRISPR-Cas guide RNA binding site is upstream of the transcribed trans-splicing element. In some embodiments, the one or more perturbation elements comprises ORF sequences, guide RNAs, siRNAs, shRNAs, miRNAs, tRNAs, snRNAs, or lncRNAs. In some embodiments, the antisense promoter is a cell-specific, tissue-specific, or organ-specific promoter. In some embodiments, the one or more perturbation elements comprises an snRNA. In some embodiments, the one or more perturbation elements comprises a guide RNA.

In another aspect, the present disclosure provides a vector comprising the nucleic acid construct described herein. In some embodiments, the vector is a viral vector. In some embodiments, the viral vector is a lentiviral vector.

In another aspect, the present disclosure provides a method of generating a barcoded nucleic acid library, comprising: delivering one or more polynucleotides into a cell, each polynucleotide comprising a sequence encoding a barcoding construct operably linked to a first promoter that is an antisense promoter, wherein the barcoding construct comprises a trans-splicing element and a barcode sequence; and a sequence encoding a perturbation element operably linked to a second promoter; generating RNA transcripts of the one or more polynucleotides delivered into the cell, wherein the RNA transcripts comprise the barcoding construct and the perturbation element; and splicing the barcoding sequence onto endogenous RNA molecules in the cell, thereby generating a barcoded library, each member of the barcoded library comprising the barcode sequence and the endogenous RNA molecules attached with the barcode sequence.

In some embodiments, each member of the barcoded library comprises a common barcode sequence. In some embodiments, further comprises delivering a plurality of polynucleotides to a plurality of cells, wherein the members of the barcoded library generated in each cell comprise a unique barcode. In some embodiments, the plurality of polynucleotides comprises sequences encoding at least 1,000 perturbation elements. In some embodiments, the plurality of cells comprise a plurality of barcoded libraries, and the method further comprises lysing the plurality of cells in a single volume. In some embodiments, the one or more polynucleotide is in a viral vector. In some embodiments, the viral vector is a lentiviral vector. In some embodiments, a strength of the first promoter is weaker than a strength of the second promoter. In some embodiments, the first promoter does not comprise a splice donor site. In some embodiments, the polynucleotide further comprises a sequence encoding a transcription terminator. In some embodiments, the transcription terminator is an antisense sequence. In some embodiments, the method further comprises eliminating non-spliced barcoding constructs. In some embodiments, the non-spliced barcoding constructs are eliminated by a CRISPR-Cas system. In some embodiments, the method further comprises sequencing the barcode sequence and the endogenous RNA. In some embodiments, one or more of the endogenous RNA molecules in the barcoded library comprises a perturbation caused by the perturbation element. In some embodiments, the polynucleotide is delivered by virus transduction. In some embodiments, the perturbation element comprise ORF sequences, mRNAs, guide RNAs, siRNAs, shRNAs, miRNAs, tRNAs, rRNAs, snRNAs, or lncRNAs. In some embodiments, the barcoding construct further comprises a reverse transcription primer binding site. In some embodiments, wherein the trans-splicing element comprises a branch point, a polypyrimidine tract, a splice acceptor sequence, or a combination thereof. In some embodiments, the trans-splicing element is a ribozyme. In some embodiments, the ribozyme comprises Tetrahymena group I intron or Azoarcus group I intron. In some embodiments, the first or the second prompter is a SV40, CMV, U6, or EF1a promoter. In some embodiments, the method further comprises generating cDNA molecules from the barcoded library. In some embodiments, the barcode sequence is flanked by at least one filter sequence. In some embodiments, the method further comprises sequencing at least a portion of the barcode sequence and at least a portion of endogenous RNA molecules attached thereto. In some embodiments, the method further comprises amplifying the barcoded library. In some embodiments, the amplification is unbiased amplification. In some embodiments, the endogenous RNA is mRNA. In some embodiments, the first promoter is a cell-specific, tissue-specific, or organ-specific promoter.

In another aspect, a method of labeling cell populations comprises delivering a plurality of polynucleotides into a plurality of cell populations, each polynucleotide comprising a sequence encoding a barcoding construct operably linked to an antisense promoter, wherein the barcoding construct comprises a trans-splicing element and a barcode sequence; in each cell, generating RNA transcripts of the polynucleotides, wherein the transcripts comprise the barcoding constructs; splicing each of the barcoding sequence onto endogenous RNA molecules in the cells, wherein cells in the same cell population comprise a common barcode sequence and the barcode sequence in each cell population is unique. In some embodiments, cells in each population are of the same lineage. In some embodiments, cells in each population are from or derived from the same species.

In another aspect, a method of performing whole-organism barcoding in a subject comprises delivering a plurality of polynucleotides into multiple types of cells in the subject, each polynucleotide comprising a sequence encoding a barcoding construct operably linked to an antisense promoter, wherein the barcoding construct comprises a trans-splicing element and a barcode sequence, and the antisense promoter is a cell-specific promoter; in each cell, generating RNA transcripts of the polynucleotides, wherein the transcripts comprise the barcoding constructs; and splicing each of the barcoding sequence onto endogenous RNA molecules in the cells, wherein cells in the same type of cells comprise a common barcode sequence and the barcode sequence in each type of cells is unique.

In some embodiments, the subject is a transgenic organism. In some embodiments, the method further comprises sequencing the barcode sequence and the endogenous RNA molecules.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows a schematic for an example trans-splicing barcoding approach using lentiviruses.

FIG. 2 shows an example method for trans-splicing barcoding.

FIG. 3 shows trans-splicing-based transcriptome barcoding is effective and robust with different approaches. “A0” stands for SV40-driven Azoarcus group I intron with a P1 helix library (5′-NNNGNN-3′). “A30” stands for SV40-driven Azoarcus group I intron with a U30 (T30 in DNA) sequence upstream of the P1 helix library, to maximize binding to the 3′ poly(A)-tail of endogenous mRNA. “AC” stands for SV40-driven Azoarcus group I intron with the wild-type P1 helix library. “EV” stands for Empty vector control. “G” stands for SV40-driven GFP control (negative control for trans-splicing, positive control for transduction, selection and expression). NTC stands for No template control. “S1” stands for SV40-driven adenovirus branch point, polypyrimidine tract and splice-acceptor (5′-tacttatcctgtcccttttttttccacagGTG-3′) (SEQ ID NO: 1). “S2” stands for SV40-driven alternative branch point, polypyrimidine tract and splice-acceptor (5′-tactaactgatatctcttctttttttttttccggaaaacagGC-3′) (SEQ ID NO:2). “TO” stands for SV40-driven Tetrahymena group I intron ribozyme with a P1 helix library (5′-G-3′). “T30” stands for SV40-driven Tetrahymena group I intron ribozyme with a U30 (T30 in DNA) sequence upstream of the P1 helix library, to maximize binding to the 3′ poly(A)-tail of endogenous mRNA. “TC” stands for SV40-driven Tetrahymena group I intron with the wild-type P1 helix library. “Wt” stands for Wt 293T cells.

FIG. 4 shows that the example trans-splicing-based transcriptome barcoding approach was quantitative.

FIG. 5 shows an two-species mixing experiment demonstrating the example approach can barcode specific cell populations.

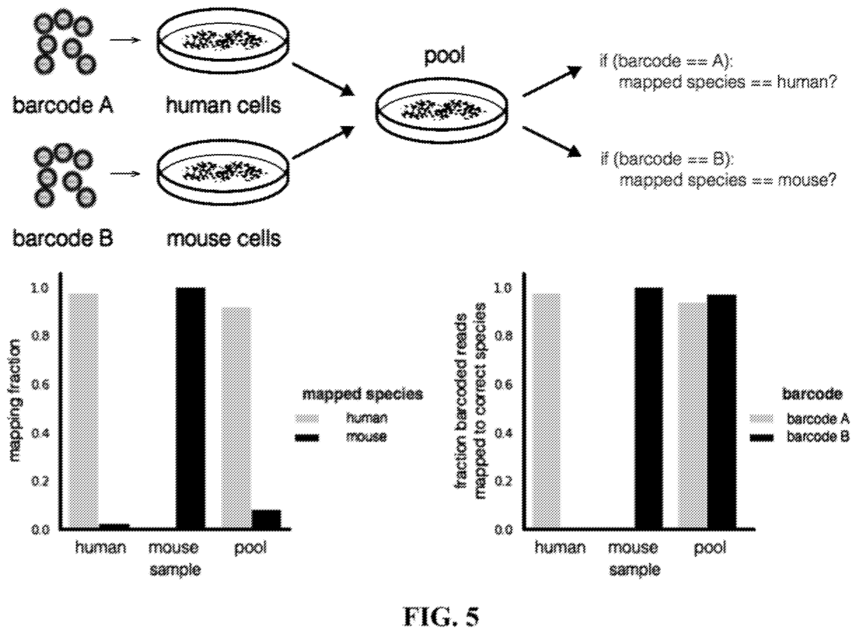

FIG. 6 shows that RNA barcoding according to an example embodiment was not perturbative in a test.

FIG. 7 shows that RNA barcoding according to an example embodiment was quantitative.

FIG. 8 demonstrates the information that may be obtained from RNA barcoding according to an example embodiment.

FIG. 9 shows an example approach for whole-organism RNA barcoding.

FIG. 10 shows an exemplary construct for RNA barcoding.

FIG. 11 shows an exemplary method of RNA barcoding using the construct in FIG. 10.

FIG. 12 shows RNA barcoding with an exemplary ORF library.

FIG. 13 shows ORF expression and barcode map validation in the RNA barcoding in FIG. 12.

The figures herein are for illustrative purposes only and are not necessarily drawn to scale.

### DETAILED DESCRIPTION OF THE EXAMPLE EMBODIMENTS

General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

Cells as described herein may be from or derived a cellular sample. The cellular sample may be made up of a collection or mixture of heterogeneous cells with different phenotypes. In some instances, a population of cells with the same phenotype can be also heterogeneous at the gene expression level. In some cases, the cells are mammalian cells, e.g., cells from or derived from a mammal such as human, rat, mouse, rabbit, monkey, baboon, chicken, bovine, porcine, ovine, canine, feline, or any other mammal of interest. The cells may be grown in a model organism (e.g., xenograft model of cancer in mice) prior to the processing and analysis described herein. The cells may be disease-free cells, diseased cells, or a mixture thereof. By “diseased” is meant any condition or disorder that damages or interferes with the normal function of a cell, tissue, or organ. In some cases, diseased cells may exhibit abnormal changes in proliferation, cell death, cell metabolism, cell signaling, immune response, replicative control, and/or motility due to environmental, genetic or epigenetic factors. In some examples, diseased cells may be tumor cells, e.g., cells derived from cancers of the colon, breast, lung, prostate, skin, pancreas, brain, kidney, endometrium, cervix, ovary, thyroid, or other glandular tissue carcinomas or melanoma, lymphoma, genetically modified cells or cells treated with mutagenic and/or cancer-causing agents, or any other cancers of interest.

In some cases, the cells herein include Cas transgenic cells. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides methods and compositions for increasing the throughput of generating sequencing libraries, e.g., libraries of barcoded of mRNA molecules and/or transcripts thereof. In general, a barcode sequence and a perturbation element (e.g., siRNA or sgRNA) may be transcribed from a single polynucleotide within a cell. The barcode sequenced may be attached to various endogenous RNA molecules by trans-splicing in the cell, thereby generating a barcoded library. In cells expressing the same perturbation element, these endogenous RNA molecules have a common barcode sequence. In some cases, each perturbation is associated with a unique barcode. Thus, the effects of a given perturbation element on the RNA molecules may be determined and correlated to the perturbation using the barcode sequence, for example by isolating and sequencing the endogenous RNA molecules comprising the barcode sequence. With the barcodes identifying the perturbations, a plurality of cells expressing multiple perturbation elements can be lysed in a single volume to generate RNA-seq libraries. The resulting barcoded libraries may map both to i) a cellular lineage, genetic perturbation, pharmacological or environmental perturbation and ii) the transcriptomic outcome of the condition(s) assayed.

In one aspect, the present disclosure provides polynucleotides for generating barcoded libraries. In general, each polynucleotide may comprise a sequence encoding a barcoding construct and a sequence encoding a perturbation element. The barcoding construct may comprise a trans-splicing element and a barcode sequence. The barcode sequence may be used for identifying the perturbation element transcribed from the same polynucleotide. In some examples, the barcoding construct is driven by an anti-sense promoter. The perturbation element may be driven by a different promoter than the one for the barcoding construct. After delivered to a cell, the polynucleotide may be integrated into the genome of the cell. The polynucleotide may be transcribed to generate barcoding construct RNA and the perturbation element RNA. The barcoding construct RNA may comprise a trans-splicing element and a barcode sequence. The trans-splicing element may attach the barcode sequence to an endogenous mRNA molecule in the cell by trans-splicing. Features (e.g., mutations, levels, etc.) of the mRNA may be determined. Such features may be correlated with a perturbation using a barcode. For example, the mRNA molecules may be correlated with the perturbation using information in the barcode. Effects of the perturbation on the mRNA molecules may be determined.

In another aspect, the present disclosure also provides for nucleic acid constructs for barcoding a plurality of cell populations. For example, the barcoding constructs comprising unique barcode sequences may be spliced on endogenous nucleic acids within cells. The cells in each population may comprise the same unique barcode, and the barcodes may be used to identify different cell populations.

In another aspect, the present disclosure includes methods of generating barcoded nucleic acid libraries. In some embodiments, the methods include delivering a polynucleotide encoding a barcoding construct and a perturbation element into a cell, producing the barcoding construct and the perturbation element in the cell. The barcoding construct may then be spliced on endogenous mRNA molecules to generate a barcoded library. Each member of the barcoded library comprises a common barcode sequence and a mRNA sequence. In some examples, a method of generating a barcoded nucleic acid library includes: delivering a polynucleotide into a cell, each polynucleotide comprising: (i) a sequence encoding a barcoding construct operably linked to a first promoter that is an antisense promoter, wherein the barcoding construct comprises a trans-splicing element and a barcode sequence, and (ii) a sequence encoding a perturbation element operably linked to a second promoter; generating RNA transcripts of the polynucleotide delivered into the cell, wherein the RNA transcripts comprise the barcoding construct and the perturbation element; and splicing the barcoding sequence onto endogenous RNA molecules in the cell, thereby generating a barcoded library, each member of the barcoded library comprising the barcode sequence and the endogenous RNA molecule attached with the barcode sequence.

In another aspect, the present disclosure further includes methods of barcoding cell populations. The methods may include delivering a plurality of polynucleotides barcoding constructs cells, producing the barcoding constructs in cells, and splicing the barcode sequences in the barcoding construct to endogenous mRNA molecules in the cells. Cells in the same population may comprise a common barcode sequence. In some examples, a method of labeling cell populations includes delivering a plurality of polynucleotides into a plurality of cell populations, each polynucleotide comprising a sequence encoding a barcoding construct operably linked to an antisense promoter, wherein the barcoding construct comprises a trans-splicing element and a barcode sequence; in each cell, generating RNA transcripts of the polynucleotides, wherein the transcripts comprise the barcoding constructs; splicing each of the barcoding sequence onto endogenous RNA molecules in the cells, wherein cells in the same cell population comprise a common barcode sequence and the barcode sequence in each cell population is unique. The barcode sequences may unique among different cell populations. For example, cells in different populations have different barcode sequences.

In some embodiments, the methods include attaching a nucleic acid barcode to trans-splicing elements, such as ribozymes or transcripts with canonical splicing features that lack a splice donor. The methods enable mapping sequenced nucleic acids (e.g., RNA) to conditions of interest. In some cases, by having unique lineage barcodes or perturbation barcode, one could harvest cells en masse and generate sequencing libraries without the need of compartments such as wells or emulsion droplets. The methods and compositions may be used for generating libraries of barcodes that maps uniquely to open reading frames (ORFs) for high-throughput gain-of-function screens, or sgRNAs for high-throughput CRISPR knockout studies, CRISPR interference (CRISPRi) or CRISPR activation (CRISPRa) screens. In a particular example, the methods may generate RNA nucleic acids comprising one or more barcodes and a sequence mapping to the genome as a result from a successful trans-splicing reaction.

In some embodiments, since barcodes are conjugated to nucleic acids (exogenous and/or endogenous) within the cell, there is no need for compartmentalization with wells or droplets. This feature significantly increases the throughput of generating sequencing libraries, and enables large screens (>1000 elements) to take place in a single dish. The methods may also enable whole-organism RNA barcoding, where RNA can be retrieved from an entire organism and mapped to a particular organ/lineage.

### Polynucleotides

Compositions provided herein include polynucleotides comprising one or more encoding sequences. In some examples, a polynucleotide comprises a sequence encoding a barcoding construct. The polynucleotide may further comprise a sequence encoding another element, such as a perturbation element. As used herein, a polynucleotide may be DNA, RNA, or a hybrid thereof, including without limitation, cDNA, mRNA, genomic DNA, mitochondrial DNA, guide RNA, siRNA, shRNA, miRNA, tRNA, rRNA, snRNA, lncRNA, and synthetic (such as chemically synthesized) DNA or RNA or hybrids thereof. In some examples, a nucleic acid is mRNA. The nucleic acid may be double-stranded or single-stranded. Where single-stranded, the nucleic acid may be the sense strand or the antisense strand. Nucleic acids can include natural nucleotides (such as A, T/U, C, and G), modified nucleotides, analogs of natural nucleotides, such as labeled nucleotides, or any combination thereof. In some examples, the polynucleotides encode the barcode constructs and the perturbation elements.

A polynucleotide may comprise one or more regulatory elements (or sequences encoding thereof), such as transcription control sequences, e.g., sequences which control the initiation, elongation and termination of transcription. Particularly important transcription control sequences are those which control transcription initiation, such as promoter, enhancer, operator and repressor sequences. In some cases, regulatory element may be a transcription terminator or a sequence encoding thereof. A transcription terminator may comprise a section of nucleic acid sequence that marks the end of a gene or operon in genomic DNA during transcription. This sequence may mediate transcriptional termination by providing signals in the newly synthesized transcript RNA that trigger processes which release the transcript RNA from the transcriptional complex. A regulatory element may be an antisense sequence. In certain case, a regulatory element may be a sense sequence.

In some cases, the polynucleotide may comprise a first promoter, a barcode construct operably linked to the first promoter, a second promoter and a perturbation element operably linked to the second promoter. In certain examples, the polynucleotide may comprise only one promoter, both the barcode construct and the perturbation element are operably linked to the promoter. In other cases, the polynucleotide may encode a barcode construct but not any perturbation element. Other examples of regulatory elements may be enhancers, e.g., WPRE; CMV enhancers; the R-U5′ segment in LTR of HTLV-I; SV40 enhancer; and the intron sequence between exons 2 and 3 of rabbit β-globin.

In some cases, the first promoter is a cell-specific, tissue-specific, or organ-specific promoter. Cell-specific, tissue-specific, or organ-specific promoters may promote transcription (e.g., transcription of the barcode) only within a certain type of cell, tissue, or organ. Such promoters may allow for expression of the barcodes in specific types of cells. Thus, different types of cells, tissues, or organs may be labeled with unique barcodes.

In some examples, the barcode constructs and perturbation elements described herein are RNA molecules. A barcode construct and a perturbation element may be encoded by different portions of a DNA polynucleotide. The barcode construct and the perturbation element may be transcribed from the polynucleotide in a cell. In such cases, the polynucleotide may be delivered to the cell. After delivery, the polynucleotide may integrate to the genome of the cell. In certain cases, the RNA barcode constructs and RNA perturbation elements may be delivered into cells, e.g., using suitable delivery vehicles such as nanoparticles or aptamers. In certain cases, the polynucleotide constructs and perturbation elements described herein are DNA molecules, are delivered via AAV, and do not integrate into the genome of the cell. In some examples, the constructs described herein are delivered to cells such that there are multiple barcodes per cell. In other examples, the multiplicity of infection is sufficiently low, such that the majority of cells have only one barcode (e.g., roughly following a Poisson distribution).

### Barcoding Constructs

The barcoding constructs herein may be used to attach barcodes to nucleic acids within cells. The barcoding constructs may be DNA, RNA, or a hybrid thereof. In some examples, the barcoding construct may be RNA. A barcoding construct may comprise one or more barcode sequences and a trans-splicing element. When delivered or produced in cells, the trans-splicing element may facilitate the attachment the barcode(s) to nucleic acids in the cells, e.g., by trans-splicing. In some cases, the barcoding constructs may also refer to nucleic acids encoding thereof.

### Barcodes

A barcode or barcode sequence described herein may comprise a sequence of nucleotides (e.g., DNA or RNA) that is used as an identifier. A barcode sequence may refer to a sequence in a barcode construct, e.g., an RNA sequence in an RNA barcode construct. A barcode sequence may also refer to a sequence in a molecule derived from the barcode sequence. For example, a barcode sequence may refer to a DNA sequence derived (e.g., by reverse transcription) from a RNA barcode construct or an RNA sequence derived (e.g., by transcription) from a DNA barcode construct.

In some cases, barcodes may be an identifier for the associated molecules (e.g., nucleic acids), nucleic acid libraries, cell populations, or an identifier of the source of an associated molecule, such as a cell-of-origin or subject. A barcode may also refer to any unique, non-naturally occurring, nucleic acid sequence that may be used to identify the originating source of a nucleic acid fragment.

A barcode may have a length of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides. In a particular example, a barcode sequence is 12 nucleotides in length. A barcode may be in single- or double-stranded form. A molecule (e.g., nucleic acid) may be labeled with multiple barcodes in combinatorial fashion, such as a barcode concatemer. In some cases, the barcodes may be RNA. In some cases, the barcode may be DNA.

In some embodiments, a barcode may be used to identify a perturbation, A barcode may be associated with a perturbation element. For example, the barcode and the perturbation element may be encoded by the same polynucleotide. The barcode and the perturbation element may be two separate molecules. Alternatively or additionally, the barcode and the perturbation element may be comprised in the same molecule. For example, the barcode and the perturbation element may be linked (e.g., with or without a linker). The barcode and the perturbation element may be produced or delivered to the same cell. In such cases, the barcode may be attached to endogenous molecules in the cell. Characteristics of the endogenous molecules may be correlated with the perturbation using the barcode.

In some embodiments, a barcode is used to identify a target molecule and/or target nucleic acid as being from a particular nucleic acid library. For example, each member in a nucleic acid library may comprise a common barcode. When there are a plurality of libraries, members in each library may comprise a unique barcode (e.g., members from different library have different barcodes) that can be used to identify the library. In these cases, multiple libraries may be pooled, processed, and/or analyzed together, e.g., in the same reaction volume. In the analysis results, information on a particular library may be extracted using the barcode, e.g., the sequence of the barcode.

In some embodiments, a barcode may be used to identify a cell population. For example, each cell in a given cell population may comprise a common barcode. The barcode may be attached to a nucleic acid molecule in the cell. For example, the barcode may be attached to an endogenous molecule (e.g., an endogenous nucleic acid or protein). In certain examples, the barcode may be attached to an exogenous molecule (e.g., a nucleic acid or protein delivered to the cell or expressed by an exogenous nucleic acid construct). In a particular example, a barcode may be attached to an endogenous mRNA molecule in a cell.

As used herein, a cell population may be a group of cells. In some embodiments, cells in a population have one or more common characteristics. Such common characteristics may include presence of one or more phenotypes, presence or absence of one or more molecules (e.g., genes or proteins).

In some examples, the common characteristics may be cell lineage. As used herein, “cell lineage” refer to cells with a common ancestry. For example, cells of the same lineage may be at the same development stage, or are developed from the same type of cell, and/or have the capability of developing into specific identifiable and/or functioning cells. Examples of cell lineages include respiratory, prostatic, pancreatic, mammary, renal, intestinal, neural, skeletal, vascular, hepatic, hematopoietic, muscle or cardiac cell lineages.

In certain cases, the common characteristic is species of origin. For example, cells in the same population are from or derived from the same species (e.g., human or mouse). Cells of different populations may be from or derived from different species. The barcode sequences may identify the species.

In certain cases, the common characteristic is individual subject origin. For example, cells in a given population are from or derived from the same individual (e.g., patient). Cells of different populations are from or derived from different individuals. The barcode sequences may identify the individuals. In some examples, the present disclosure includes a plurality of cell populations, each cell in the populations comprising a barcoded nucleic acid molecule comprising a barcoded sequence, a trans-splicing element, and an endogenous mRNA, wherein the barcoded nucleic acid molecules in each population have a common barcode. The barcode may be unique, e.g., barcoded nucleic acid molecules from different populations comprise different barcodes.

In some cases, a barcode may be used for identifying a sample. For example, cells or molecules (e.g., nucleic acids) from or derived from the same sample may comprise a common barcode. Barcodes in different samples may be unique (different from one another), such that they are capable of identifying the samples. Examples of samples that can be identified by the barcode include a biological sample, cells, cell lysates, blood smears, cyto-centrifuge preparations, cytology smears, tissue biopsies (e.g., tumor biopsies), fine-needle aspirates, and/or tissue sections (e.g., cryostat tissue sections and/or paraffin-embedded tissue sections).

In certain embodiments, a barcode may identify the type of nucleic acids molecules. For example, all DNA molecules may comprise a first common barcode sequence and all RNA molecules or cDNA molecules generated from RNA molecules may comprise a second common barcode sequence, which is different from the first common barcode sequence. In some cases, a barcode may identify the individual discrete volume. A barcode may further include an identifier specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

A cell population may comprise at least 10, at least 100, at least 200, at least 500, at least 1000, at least 2000, at least 5000, at least 10⁴, at least 10⁵, at least 10⁶, at least 10⁷, at least 10⁸, at least 10⁹, at least 10¹⁰, at least 10¹¹, at least 10¹², at least 10¹³, or at least 10¹⁴ cells. A plurality of cell populations, e.g., at least 2, at least 3, at least 4, at least 5, at least 6, at least 7, at least 8, at least 9, at least 10, at least 15, at least 20, at least 30, at least 40, or at least 50 cell populations may be barcoded with the methods and compositions herein.

The attachment between a barcode and its associated molecule (e.g., the endogenous RNA) may be direct (for example, covalent or noncovalent binding of the barcodes to the target molecule) or indirect (for example, via an additional molecule). Such indirect attachments may, for example, include a barcode bound to a specific-binding agent that recognizes a target molecule. Nucleic acid molecules may be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool.

In some cases, the number of distinct barcodes may be greater than the number of cells or cell populations into which the polynucleotides encoding the barcode sequences are designed to be delivered. For example, the number of distinct barcode sequences may be at least 2-fold, at least 3-fold, at least 4-fold, at least 5-fold, at least 6-fold, at least 7-fold, at least 8-fold, at least 9-fold, at least 10-fold, at least 10² fold, at least 10³ fold, at least 10⁴ fold, at least 10⁵ fold, at least 10⁶ fold, at least 10⁷ fold, at least 10⁸ fold, or greater than the number of cells or cell populations into which the polynucleotides encoding the barcode sequences are designed to be delivered. In some cases, the number of barcodes is greater than the number of cells or cell populations into which the polynucleotides encoding the barcode sequences are designed to be delivered, such that the minimum pairwise Levenshtein distance between all barcodes is 3, allowing the barcodes to be error corrected. In other cases, the number of barcodes is designed such that the minimum pairwise Levenshtein distance between all barcodes is 2, allowing barcode sequencing errors to be detected. In some cases, the number of barcodes is designed such that the minimum pairwise Levenshtein distance between all barcodes is between 20 and 1, between 15 and 1, between 10 and 1, between 5 and 1, for example, 1, 2, 3, 4, 5, 6, 7, 8, 9, or 10.

### Filter Sequences

The barcode sequence may be flanked by one or more filter sequences. In some cases, the filter sequence(s) are known. They may be sequenced together with the barcode sequence. When analyzing the sequence reads, the filter sequence(s) may be used to locate or identify the barcode sequences in the sequence reads. In some cases, one end of a barcode sequence is flanked with a filter sequence. In certain cases, both ends of a barcode sequence are flanked with filter sequences. In some cases, a filter sequence may directly flank a barcode sequence. In certain cases, there is an intervening sequence between a filter sequence and a barcode sequence. A filter sequence may be 1, 2, 3, 4, 5, 6, 7, 8, 9, 10, 11, 12, 13, 14, 15, 16, 17, 18, 19, 20, or more nucleotides in length. In one example, a barcode sequence (shown as a stretch of 12 Ns) flanked with filter sequences (underlined) is GGCGANNNNNNNNNNNNCCTA.

### Trans-Splicing Element

The barcoding constructs herein may further comprise one or more trans-splicing elements. The term “trans-splicing” as used herein refers to a form of genetic manipulation wherein a nucleic acid sequence of a first polynucleotide is co-linearly linked to or inserted co-linearly into the sequence of a second polynucleotide, e.g., in a manner that retains the 3′-5′ phosphodiester linkage between the polynucleotides. In some examples, trans-splicing may join exons contained on separate, non-contiguous RNA molecules, e.g., RNAs from different genes. Trans-splicing may include trans-splicing of RNA, trans-splicing at the level of translation and post-translational trans-splicing. In some cases, trans-splicing may be direct trans-splicing, e.g., a trans-splicing reaction that requires a specific species of RNA or DNA as a substrate for the trans-splicing reaction (that is, a specific species of RNA or DNA in which to splice the transposed sequence). Directed trans-splicing may target more than one RNA or DNA species if the enzymatic nucleic acid molecule is designed to be directed against a target sequence present in a related set of RNA or DNA sequences.

A trans-splicing element may be linked with a barcode sequence. For example, a trans-splicing element and a barcode sequence may be in the same nucleic acid molecule. Upon transcription of the nucleic acid molecule, the barcode may be present in any fusion transcripts generated via trans-splicing in the cell. The trans-splicing element may facilitate the attachment of the barcode to another nucleic acid by trans-splicing.

In some embodiments, a trans-splicing element is a spliceosome-mediated trans-splicing element. The spliceosome-mediated trans-splicing element may include a splice acceptor, a splice donor, or a splice acceptor and a splice donor. In spliceosome-recognized trans-splicing elements that include a splice acceptor, the splice acceptor may include a branchpoint, a polypyrimidine tract, and a 3′ splice site. In some cases, the trans-splicing element does not comprise any splice donor. For example, a trans-splicing element may comprise one or more of: a branch point (BP), polypyrimidine tract (PPT), and a splice acceptor sequence. In one example, a trans-splicing element comprises, in a 5′ to 3′ orientation, a branch point (BP), polypyrimidine tract (PPT), and a splice acceptor sequence.

Not being bound by any particular theory, in some embodiments, a trans-splicing reaction may be characterized as follows. Introns are removed from primary transcripts by cleavage at conserved sequences called splice sites. These sites are found at the 5′ and 3′ ends of introns. In some cases, the intronic RNA sequence that is removed begins with the dinucleotide (e.g., GU) at its 5′ end, and ends with dinucleotide (e.g., AG) at its 3′ end. The consensus sequences surrounding the splice sites (e.g., a splice donor site at the 5′ end of intron and a splice acceptor site at the 3′ end of the intron) are important, because changing one of the conserved nucleotides may result in inhibition of splicing. Upstream (5′-ward) from the AG in the splice acceptor site is a region high in pyrimidines (C and U) referred to as the polypyrimidine tract (PPT). Another important sequence occurs at what is called the branch point, located upstream (e.g., anywhere from 18 to 40 nucleotides upstream) from the 3′ end of an intron. In some cases, the branch point may contain an adenine, but it is otherwise loosely conserved. For example, a branch point may comprise the sequence YNYYRAY, where Y indicates a pyrimidine, N denotes any nucleotide, R denotes any purine, and A denotes adenine. The splice donor site may be more compact than the splice acceptor site and may have the consensus sequence AGAGURAGU. In addition to consensus sequences at their splice sites, eukaryotic genes may also contain exonic splicing enhancers (ESEs) and intronic splicing enhancers (ISEs). These sequences, which may help position the splicing apparatus, may be found in the exons of genes and bind proteins that recruit splicing machinery to the correct site. The splicing process occurs in organelles called spliceosomes. Pre-mRNAs (or hnRNA) contain sequence elements including a 5′ splice donor site, branch point, a polypyrimidine tract and a 3′ splice acceptor site recognized and utilized during spliceosome assembly. In some cases, a splice acceptor sequence may follow the polypyrimidine tract. In one example, a splice acceptor may have the sequence of YAGG.

The splice site in the trans-splicing element may be a promiscuous splice site. A promiscuous splice site may be designed to permit non-specific trans-splicing to the target RNA (e.g., pre-mRNA sequence). Inclusion of a promiscuous splice site in the trans-splicing element may increase the trans-splicing efficiency and uniform labeling of different mRNAs in the transduced target cell. Increasing the promiscuity of the splice site may be achieved, e.g., by modifying the three-dimensional structure and/or sequence of branch point and/or pyrimidine tract sequences, or by including one or more additional splice sites and/or regulatory elements such that they are more efficient splicing elements. In certain aspects, a splice leader sequence (e.g., which mimics or is complementary to at least a portion of the spliceosome snRNA, such as a U1, U2, U4, U5, U7 and/or U6 snRNA) is included in a splice donor or splice acceptor trans-splicing element to increase promiscuous trans-splicing activity. According to one embodiment, a splice acceptor site sequence and/or a splice donor site sequence is included in the structure of a snRNA, such as a modified U7 snRNA, U5 snRNA and/or the like. In some examples, the construct herein comprises a U2 snRNA. Examples of snRNAs (e.g., U2 snRNA) include those described in van der Feltz C, et al., Crit Rev Biochem Mol Biol. 2019 October; 54(5):443-465; and Shi Y. J Mol Biol. 2017 Aug. 18; 429(17):2640-2653.

According to certain embodiments, the trans-splicing element includes an RNA polymerase pause or termination site in a splice donor- and/or splice-acceptor-containing trans-splicing element to increase the efficiency of the trans-splicing reaction. Alternatively, or additionally, promiscuity of the trans-splicing element is increased by excluding sequences in the trans-splicing element which could interact with specific pre-mRNA sequences. In certain aspects, a pre-mRNA target binding domain is included in the trans-splicing element to facilitate labeling a specific sub-population of mRNAs, e.g., a fraction of RNAs having a specific conserved nucleotide sequence. Such trans-splicing elements with mRNA binding domains have been used to correct genetic defects in mRNA splicing and delivery of suicidal trans-spliced constructs to cancer cells. In certain embodiments, the splice site in the trans-splicing element may be a sequence specific splice site.

In certain embodiments, a trans-splicing element may serve as both a trans-splicing element and a barcode. For example, a trans-splicing element may be modified by introducing point mutations which result in the element having a barcode; the mutations do not affect the functionality of the trans-splicing element. The developed plurality/library of functional trans-spliced elements could be used as both trans-splicing element and barcode.

A trans-splicing element may further include a regulatory sequence such as a spliced leader sequence, splice enhancer, snRNA-interaction domain, and other sequences which facilitates/promotes trans-splicing in cells.

### Ribozyme

In some embodiments, the trans-splicing element may comprise a ribozyme. The term “ribozyme” refers to an RNA molecule capable of catalyzing a biochemical reaction. Ribozymes may catalyze various RNA processing functions, such as splicing, viral replication, and tRNA biosynthesis. Ribozymes may be self-cleaving. In some embodiments, ribozymes may function in protein synthesis, catalyzing the linking of amino acids in the ribosome. Examples of ribozymes include the HDV ribozyme, the Lariat capping ribozyme (formally called GIR1 branching ribozyme), the glmS ribozyme, group I and group H self-splicing introns, the hairpin ribozyme, the hammerhead ribozyme, various rRNA molecules, RNase P, the twister ribozyme, the VS ribozyme, the pistol ribozyme, and the hatchet ribozyme. In some cases, the ribozyme allows for a barcode and reverse-transcription handle to be ligated to endogenous transcripts via trans-splicing.

In some embodiments, the ribozyme may be Group I introns. For example, Group I introns include the self-splicing intron in the pre-ribosomal RNA of the ciliate Tetrahymena thermophilia. Further examples of group I introns interrupt genes for rRNAs, tRNAs and mRNAs in a wide range of organelles and organisms. Not being bound by any theory, in some examples, Group I introns perform a splicing reaction by a two-step transesterification mechanism. The reaction is initiated by a nucleophilic attack of the 3′-hydroxyl group of an exogenous guanosine cofactor on the 5′-splice site. Subsequently, the free 3 ‘-hydroxy I of the upstream exon performs a second nucleophilic attack on the 3’-splice site to ligate both exons and release the intron. Substrate specificity of group I introns is achieved by an Internal Guide Sequence (IGS). The catalytically active site for the transesterification reaction resides in the intron, which can be re-engineered to catalyze reactions in trans. In one example, the ribozyme is Tetrahymena group I intron. In another example, the ribozyme is Azoarcus group I intron. Other ribozymes may also be ribozymes from Pneumocystis, Didymium iridis (DiGIR2), and Fuligo (e.g., Fse.L569 and Fse.L1898).

Other RNA processing or modifications approaches may also be used for the barcoding process. Examples of such RNA processing or modification approaches include exon shuffling, template-switching, sequence-specific oligonucleotide trans-splicing, CRISPR-mediated recombination, and/or the like.

### Regulatory Elements

The barcoding construct may further comprise one or more regulatory elements, such as transcription control sequences, translation control sequences, origins of replication. In cases where the barcoding construct is RNA, it may also comprise an element for regulating or controlling reverse transcription. In some cases, the barcoding construct comprises a reverse transcription primer binding site. In certain cases, the barcoding construct may comprise a reverse transcription initiation sequence, a reverse transcription termination sequence, or both. The barcoding construct may also comprise one or more sequencing primer binding sites.

### Perturbation Elements

The polynucleotide herein may comprise a sequence coding one or more perturbation elements. A perturbation element may be a nucleic acid or polypeptide molecule capable of modulating, blocking or hindering, enhancing, altering cellular functions such as transcription factor activation, localization of nucleotides, polypeptides, or combinations thereof within areas of a cell (e.g. modulating localization into an cellular organelle), a protein degradation through a cellular protein degradation pathway, including though the action of proteases, proteasomes, and lysosomal degradation, interactions between a protein, such as a kinase, and ligand in a signal transduction cascade, translational efficiency, promoter activities, or any combination thereof. Examples of the perturbation elements include genomic DNA, cDNA (e.g., for overexpression), genes, ORFs, mRNA, guide RNA, siRNA, shRNA, miRNA, tRNA, rRNA, snRNA, lncRNA, polypeptides or proteins (e.g., enzymes or transcription factors), DNA encoding thereof, or any combination thereof. In some cases, a perturbation element may comprise UTR sequences (e.g. 3′ UTR sequences or 5′ UTR sequences). In some examples, the perturbation elements are snRNAs (e.g., U2 snRNAs). In some examples, the perturbation elements are guide RNAs, e.g., single guide RNAs.

The polynucleotides delivered in cells may comprise coding sequences for a plurality of perturbation elements, e.g., at least 5, at least 10, at least 50, at least 100, at least 200, at least 400, at least 600, at least 800, at least 1,000, at least 1,200, at least 1,400, at least 1,600, at least 1,800, at least 2,000, at least 2,500, at least 3,000, at least 4,000, or at least 5,000 perturbation elements. In some cases, the coding sequence of each of the perturbation element is linked with a unique barcode sequence or a sequence encoding thereof.

### Guide Molecules

In some embodiments, the perturbation elements may be guide molecules in CRISPR-Cas systems. As used herein, the term “guide sequence” and “guide molecule” in the context of a CRISPR-Cas system comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In some embodiments, the guide sequence is an RNA sequence of between 10 to 50 nt in length, but more particularly of about 20 to 30 nt advantageously about 20 nt, 23 to 25 nt or 24 nt. The guide sequence is selected so as to ensure that it hybridizes to the target sequence. This is described more in detail below. Selection can encompass further steps which increase efficacy and specificity.

In certain embodiments, a guide molecule comprises (1) a guide sequence capable of hybridizing to a target locus and (2) a tracr mate or direct repeat sequence whereby the direct repeat sequence is located upstream (e.g., 5′) from the guide sequence. In a particular embodiment the seed sequence (i.e., the sequence essential critical for recognition and/or hybridization to the sequence at the target locus) of the guide sequence is approximately within the first 10 nucleotides of the guide sequence. In a particular embodiment the guide molecule comprises a guide sequence linked to a direct repeat sequence, wherein the direct repeat sequence comprises one or more stem loops or optimized secondary structures.

In particular embodiments, use is made of a truncated guide (tru-guide), i.e., a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

A guide molecule may form a complex with CRISPR-Cas protein. In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008; and Makarova et al. “Evolutionary classification of CRISPR-Cas systems: a burst of class 2 and derived variants” Nature Reviews Microbiology, 18:67-81 (February 2020). Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cas12a, Cas12b, Cas12c, Cas12d, CasX, CasY, Cas13a, Cas13b, Cas13c, Cas13d, homologues thereof, or modified versions thereof.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

Examples of perturbation elements include those used for introducing genetic variations using CRISPR-Cas systems, including those described in Shalem O, et al., High-throughput functional genomics using CRISPR-Cas9, Nat Rev Genet. 2015 May; 16(5):299-311; Sanjana N E, et al., Genome-scale CRISPR pooled screens, Anal Biochem. 2017 Sep. 1; 532:95-99; Miles L A, et al., Design, execution, and analysis of pooled in vitro CRISPR/Cas9 screens, FEBS J. 2016 September; 283(17):3170-80; Ford K, et al., Functional Genomics via CRISPR-Cas, J Mol Biol. 2019 Jan. 4; 431(1):48-65.

Examples of perturbation elements include guide molecules used in CRISPR-Cas systems with additional functional domains and proteins. Examples of the systems include base editors (e.g., those described in Cox D B T, et al., RNA editing with CRISPR-Cas13, Science. 2017 Nov. 24; 358(6366):1019-1027; Abudayyeh O O, et al., A cytosine deaminase for programmable single-base RNA editing, Science 26 Jul. 2019: Vol. 365, Issue 6451, pp. 382-386; Gaudelli N M et al., Programmable base editing of A⋅T to G⋅C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19; 533(7603):420-4; Jordan L. Doman et al., Evaluation and minimization of Cas9-independent off-target DNA editing by cytosine base editors, Nat Biotechnol (2020)), prime editing systems (e.g., those described in Anzalone A V et al., Search-and-replace genome editing without double-strand breaks or donor DNA, Nature. 2019 Oct. 21. doi: 10.1038/s41586-019-1711-4), CAST systems (e.g., those described in Strecker J et al., RNA-guided DNA insertion with CRISPR-associated transposases. Science. 2019 Jul. 5; 365(6448):48-53; Klompe S E, et al., Transposon-encoded CRISPR-Cas systems direct RNA-guided DNA integration. Nature. 2019 July; 571(7764):219-225).

In some embodiments, the nucleic acid construct may comprise a guide RNA. Such constructs may be used for nucleic acid (e.g., RNA) barcoding. For examples, the construct may comprise a modified CROPseq vector. The construct may pair transcriptomic signatures of cells to their corresponding guides.

In some cases, the construct may be used for a Cas KO screen, where the modified vector is delivered to cells that express or can conditionally or inducibly express Cas protein. For example, the construct may be used for Cas9 KO screen, Cas13 KO screen, Cas12 KO screen, or KO screen with other types of Cas proteins. In some cases, the screen is a Cas13d KO screen, where the scaffold precedes the guide, so a reverse transcription handle may be selected 3′ to the guide. The vector may be designed to have a type IIS cloning site (BsmbI or BbsI for example) in order to clone in a guide library with golden gate assembly. The downstream library construction may entail a reverse transcription, amplification, tagmentation, step in linear amplification, and finally an index PCR to make a sequencing library (e.g., Illumina compatible sequencing library).

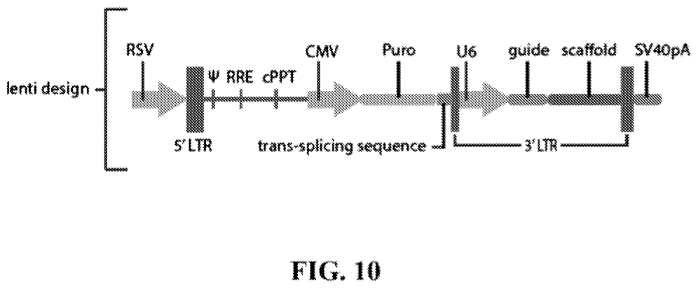

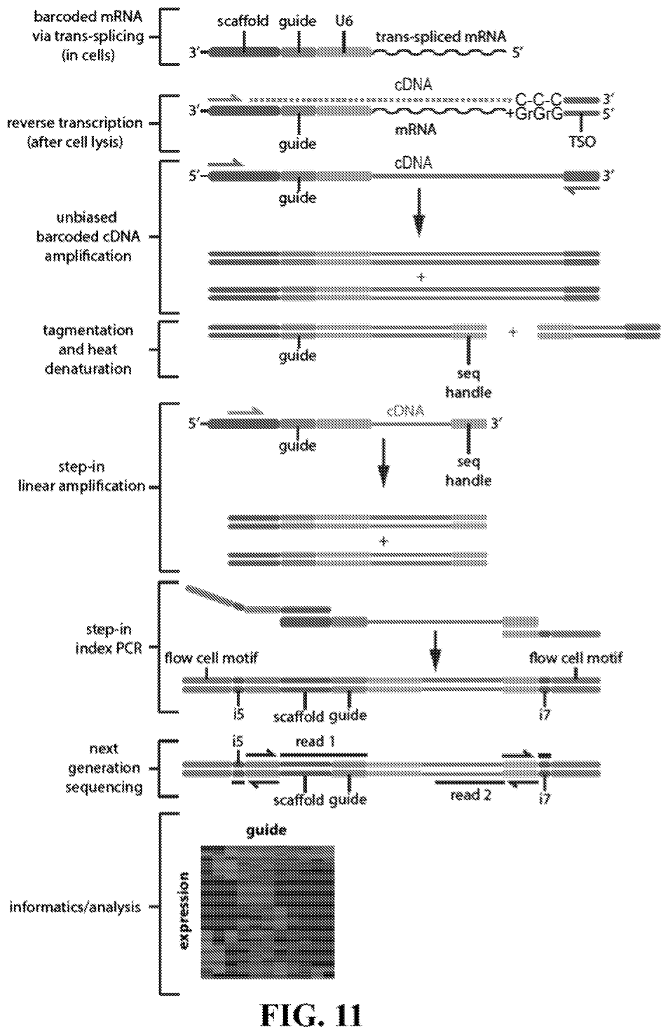

An example of such construct is shown in FIG. 10, and an exemplary method of RNA barcoding using the construct is shown in FIG. 11. In some cases, upon transduction, the U6 cassette is copied upstream, to drive guide expression, meanwhile a pol II transcript is transcribed from CMV, allowing for puro resistance and trans-splicing based transcriptome barcoding.

### Promoters

The polynucleotides may comprise one or more promoters. A promoter or promoter region refers to a nucleic acid sequence that directs the transcription of a operably linked sequence into mRNA. The promoter or promoter region typically provide a recognition site for RNA polymerase and the other factors necessary for proper initiation of transcription when a sequence operably linked to a promoter is controlled or driven by the promoter. The promoter(s) may drive the transcription of the barcoding construct and/or other elements encoded by the polynucleotides, such as the perturbation elements. In some cases, a promoter does not have any splice donor sequence. Alternatively or additionally, a promoter does not have any splicing acceptor sequence.

In the polynucleotide, a barcode construct encoding sequence may be operably linked with a promoter. In some examples, a construct encoding sequence may be operably linked to a first promoter and a sequence encoding another element may be operably linked to a second promoter. The first and the second promoters may be the same. Alternatively, the first and the second promoters may be different promoters.

In some cases, the promoter may be an anti-sense promoter. An anti-sense promoter may be upstream of the sequence controlled by the promoter in the 3′ to 5′ direction. In cases where the polynucleotide is double-stranded, an antisense promoter joins at the 5′ of the sequence controlled by the promoter in the template strand. In some cases, barcoding constructs may be driven by an anti-sense promoter. Such design may prevent undesired 3′ LTR->5′ LTR transcription and cis-splicing. For example, without such design, undesired transcription that occurs from the 3′ LTR to the 5′ LTR may lead to cis-splicing.

In certain cases, the promoter may be a sense promoter. A sense promoter may be upstream of the sequence controlled by the promoter in the 5′ to 3′ direction. In cases where the polynucleotide is double-stranded, a sense promoter joins at the 3′ of the sequence controlled by the promoter in the template strand. When a polynucleotide has multiple coding sequences, some of the coding sequence may be controlled by sense promoters and some by anti-sense promoters. For example, a polynucleotide may comprise a sequence coding of a barcoding construct controlled by an anti-sense promoter and a sequence coding of another element (e.g., a perturbation element) by a sense promoter. The anti-sense promoter may not comprise a splice donor site.

In some cases, the promoter may be a constitutive promoter, e.g., U6 and H1 promoters, retroviral Rous sarcoma virus (RSV) LTR promoter, cytomegalovirus (CMV) promoter, SV40 promoter, dihydrofolate reductase promoter, β-actin promoter, phosphoglycerol kinase (PGK) promoter, ubiquitin C, U5 snRNA, U7 snRNA, tRNA promoters or EF1α promoter. In certain cases, the promoter may be a tissue-specific promoter and may direct expression primarily in a desired tissue of interest, such as muscle, neuron, bone, skin, blood, specific organs (e.g. liver, pancreas), or particular cell types (e.g. lymphocytes). Examples of tissue-specific promoters include Ick, myogenin, or thy1 promoters. In some embodiments, the promoter may direct expression in a temporal-dependent manner, such as in a cell-cycle dependent or developmental stage-dependent manner, which may or may not also be tissue or cell-type specific. In certain cases, the promoter may be an inducible promoter, e.g., can be activated by a chemical such as doxycycline.

The promoters may have suitable strengths for their desired functions. The activity or strength of a promoter may be measured in terms of the amounts of RNA it produces, or the amount of protein accumulation in a cell or tissue, relative to a promoter whose transcriptional activity has been previously assessed. In some examples, the relative strength of promoter activity may be determined, either by means of replica plating onto culture media containing increasing concentrations of antibiotic, or by employing “crippled” antibiotic genes as the selective marker in the transposon cassette. For example, a modified neomycin resistance gene can be employed where, in order to get resistance to the antibiotic, a high-level of expression of the neomycin resistance gene is required. In one embodiment the crippled selectable marker is a neomycin resistance (Neon) sequence in which amino acid residue 182 (Glu) is mutated to Asp. (Yanofsky, et al., (1990) PNAS USA 87:3435-39). Use of such crippled selectable markers improves the strength of the selection, because more of the enzyme is required to produce antibiotic resistance.

The polynucleotide may comprise promoters of different strength. For example, the polynucleotide may comprise a first promoter that weaker, e.g., having from 10% to 30%, from 20% to 40%, from 30% to 50%, from 40% to 60%, from 50% to 70%, from 60% to 80%, from 70% to 90%, from 80% to 99%, such as about 10%, about 20%, about 30%, about 40%, about 50%, about 60%, about 70%, about 80%, or about 90% of the strength of a second promoter on the polynucleotide. In one example, the polynucleotide comprises a first promoter operably linked to a barcoding construct and a second promoter operably linked to a perturbation element, wherein the first promoter is weaker than the second promoter.

In some cases, the promoters may be cell-specific, tissue-specific, or organ-specific promoters. Example of cell-specific, tissue-specific, or organ-specific promoters include promoter for creatine kinase, (for expression in muscle and cardiac tissue), immunoglobulin heavy or light chain promoters (for expression in B cells), smooth muscle alpha-actin promoter. Exemplary tissue-specific promoters for the liver include HMG-COA reductase promoter, sterol regulatory element 1, phosphoenol pyruvate carboxy kinase (PEPCK) promoter, human C-reactive protein (CRP) promoter, human glucokinase promoter, cholesterol 7-alpha hydroylase (CYP-7) promoter, beta-galactosidase alpha-2,6 sialyltransferase promoter, insulin-like growth factor binding protein (IGFBP-1) promoter, aldolase B promoter, human transferrin promoter, and collagen type I promoter. Exemplary tissue-specific promoters for the prostate include the prostatic acid phosphatase (PAP) promoter, prostatic secretory protein of 94 (PSP 94) promoter, prostate specific antigen complex promoter, and human glandular kallikrein gene promoter (hgt-1). Exemplary tissue-specific promoters for gastric tissue include H+/K+-ATPase alpha subunit promoter. Exemplary tissue-specific expression elements for the pancreas include pancreatitis associated protein promoter (PAP) include elastase 1 transcriptional enhancer, pancreas specific amylase and elastase enhancer promoter, and pancreatic cholesterol esterase gene promoter. Exemplary tissue-specific promoters for the endometrium include the uteroglobin promoter. Exemplary tissue-specific promoters for adrenal cells include cholesterol side-chain cleavage (SCC) promoter. Exemplary tissue-specific promoters for the general nervous system include gamma-gamma enolase (neuron-specific enolase, NSE) promoter. Exemplary tissue-specific promoters for the brain include the neurofilament heavy chain (NF-H) promoter. Exemplary tissue-specific promoters for lymphocytes include the human CGL-1/granzyme B promoter, the terminal deoxy transferase (TdT), lambda 5, VpreB, and 1ck (lymphocyte specific tyrosine protein kinase p561ck) promoter, the humans CD2 promoter and its 3′transcriptional enhancer, and the human NK and T cell specific activation (NKG5) promoter. Exemplary tissue-specific promoters for the colon include pp60c-src tyrosine kinase promoter, organ-specific neoantigens (OSNs) promoter, and colon specific antigen-P promoter. Exemplary tissue-specific promoters for breast cells include the human alpha-lactalbumin promoter. Exemplary tissue-specific promoters for the lung include the cystic fibrosis transmembrane conductance regulator (CFTR) gene promoter.

Examples of cell-specific, tissue-specific, or organ-specific promoters may also include those used for expressing the barcode or other transcripts within a particular plant tissue (See e.g., International Patent Publication No. WO 2001/098480A2, “Promoters for regulation of plant gene expression”). Examples of such promoters include the lectin (Vodkin, Prog. Clinc. Biol. Res., 138:87-98 (1983); and Lindstrom et al., Dev. Genet., 11:160-167 (1990)), corn alcohol dehydrogenase 1 (Dennis et al., Nucleic Acids Res., 12:3983-4000 (1984)), corn light harvesting complex (Becker, Plant Mol Biol., 20(1): 49-60 (1992); and Bansal et al., Proc. Natl. Acad. Sci. U.S.A., 89:3654-3658 (1992)), corn heat shock protein (Odell et al., Nature (1985) 313:810-812; and Marrs et al., Dev. Genet., 14(1):27-41 (1993)), small subunit RuBP carboxylase (Waksman et al., Nucleic Acids Res., 15(17):7181 (1987); and Berry-Lowe et al., J. Mol. Appl. Genet., 1(6):483-498 (1982)), Ti plasmid mannopine synthase (Ni et al., Plant Mol. Biol., 30(1):77-96 (1996)), Ti plasmid nopaline synthase (Bevan, Nucleic Acids Res., 11(2):369-385 (1983)), petunia chalcone isomerase (Van Tunen et al., EMBO J., 7:1257-1263 (1988)), bean glycine rich protein 1 (Keller et al., Genes Dev., 3:1639-1646 (1989)), truncated CaMV 35s (Odell et al., Nature (1985) 313:810-812), potato patatin (Wenzler et al., Plant Mol. Biol., 13:347-354 (1989)), root cell (Yamamoto et al., Nucleic Acids Res., 18:7449 (1990)), maize zein (Reina et al., Nucleic Acids Res., 18:6425 (1990); Kriz et al., Mol. Gen. Genet., 207:90-98 1987; Wandelt and Feix, Nucleic Acids Res., 17:2354 (1989); Langridge and Feix, Cell, 34:1015-1022 (1983); and Reina et al., Nucleic Acids Res., 18:7449 (1990)), globulin-1 (Belanger et al., Genetics, 129:863-872 (1991)), α-tubulin, cab (Sullivan et al., Mol. Gen. Genet., 215:431-440 (1989)), PEPCase (Cushman et al., Plant Cell, 1(7):715-25 (1989)), R gene complex-associated promoters (Chandler et al., Plant Cell, 1: 1175-1183 (1989)), and chalcone synthase promoters (Franken et al., EMBO J., 10:2605-2612, 1991)). Examples of tissue-specific promoters also include those described in the following references: Yamamoto et al., Plant J (1997) 12(2):255-265; Kawamata et al., Plant Cell Physiol. (1997) 38(7):792-803; Hansen et al., Mol. Gen Genet. (1997) 254(3):337); Russell et al., Transgenic Res. (1997) 6(2):157-168; Rinehart et al., Plant Physiol. (1996) 112(3):1331; Van Camp et al., Plant Physiol. (1996) 112(2):525-535; Canevascini et al., Plant Physiol. (1996) 112(2):513-524; Yamamoto et al., Plant Cell Pkysiol. (1994) 35(5):773-778; Lam, Results Probl. Cell Differ. (1994) 20:181-196; Orozco et al., Plant Mol. Biol. (1993) 23(6):1129-1138; Matsuoka et al., Proc Natl. Acad. Sci. USA (1993) 90(20):9586-9590; and Guevara-Garcia et al., Plant J. (1993) 4(3):495-505; maize phosphoenol carboxylase (PEPC) has been described by Hudspeth & Grula (Plant Molec Biol 12: 579-589 (1989)); leaf-specific promoters such as those described in Yamamoto et al., Plant J. (1997) 12(2):255-265; Kwon et al., Plant Physiol. (1994) 105:357-367; Yamamoto et al., Plant Cell Physiol. (1994) 35(5):773-778; Gotor et al., Plant J. (1993) 3:509-518; Orozco et al., Plant Mol. Biol. (1993) 23(6):1129-1138; and Matsuoka et al., Proc. Natl. Acad. Sci. USA (1993) 90(20):9586-9590.

### Vectors

The polynucleotides herein may be in a vector. In some cases, a vector comprises a polynucleotide, the polynucleotide comprising a sequence encoding a barcoding construct operably linked to a first promoter that is an antisense promoter, wherein the barcoding construct comprises a trans-splicing element and a barcode sequence.

The vector may be used for delivering the polynucleotide to cells and/or control the expression of the polynucleotide. A vector refers to a nucleic acid molecule capable of transporting another nucleic acid to which it has been linked. A vector may be a replicon, such as a plasmid, phage, or cosmid, into which another DNA segment may be inserted so as to bring about the replication of the inserted segment. Generally, a vector is capable of replication when associated with the proper control elements. Examples of vectors include nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. A vector may be a plasmid, e.g., a circular double stranded DNA loop, into which additional DNA segments can be inserted, such as by standard molecular cloning techniques.

Certain vectors may be capable of directing the expression of genes to which they are operatively-linked. Such vectors are referred to herein as “expression vectors.” Common expression vectors of utility in recombinant DNA techniques are often in the form of plasmids. A vector may be a recombinant expression vector that comprises a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. As used herein, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

A vector may be a viral vector, wherein virally-derived DNA or RNA sequences are present in the vector for packaging into a virus. Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and thereby are replicated along with the host genome.

In some embodiments, vectors herein are lentiviral vectors. For example, the vectors may be packaged in lentiviruses. The vectors may be delivered into cells that are transduced by the lentiviruses. Within the cells, the vectors or portions thereof may be integrated into the genome of the cells. A lentiviral vector may be a vector derived from at least a portion of a lentivirus genome, including a self-inactivating lentiviral vector. Lentiviral vectors are a type of retrovirus that can infect both dividing and nondividing cells because their preintegration complex (virus “shell”) can get through the intact membrane of the nucleus of the target cell. Examples of lentivirus vectors that may be used in the clinic include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The lentiviral vectors may include sequences form the 5′ and 3′ LTRs of a lentivirus. In some examples, the vectors include the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. The vectors may contain deletions of the regulatory elements in the downstream long-terminal-repeat sequence, eliminating transcription of the packaging signal that is required for vector mobilization. As such, the vector region may include an inactivated or self-inactivating 3′ LTR. The 3′ LTR may be made self-inactivating. For example, the U3 element of the 3′ LTR may contain a deletion of its enhancer sequence, such as the TATA box, Sp1 and NF-kappa B sites. As a result of the self-inactivating 3′ LTR, the provirus that is integrated into the host cell genome will comprise an inactivated 5′ LTR. Optionally, the U3 sequence from the lentiviral 5′ LTR may be replaced with a promoter sequence in the viral construct. This may increase the titer of virus recovered from the packaging cell line. An enhancer sequence may also be included. In certain aspects, the barcoded trans-splicing viral construct is a non-integrating lentiviral construct, where the construct does not integrate by virtue of having a defective (e.g., by site-specific mutation) or absent integrase gene.

### Delivery of Polynucleotides

Polynucleotides herein may be delivered to cell using suitable methods. In some embodiments, the polynucleotides may be packaged in viruses or particles, or conjugated to a vehicle for delivering into cells.

In some embodiments, the methods include packaging the polynucleotides in viruses and transducing cell with the viruses. Transduction or transducing herein refers to the delivery of a polynucleotide molecule to a recipient cell either in vivo or in vitro, by infecting the cells with a virus carrying that polynucleotide molecule. The virus may be a replication-defective viral vector. In some examples, the viruses may be virus (e.g., retroviruses, replication defective retroviruses, adenoviruses, replication defective adenoviruses, and adeno-associated viruses (AAVs)).

In some examples, the viruses are lentiviruses. Lentiviruses are complex retroviruses that have the ability to infect and express their genes in both mitotic and post-mitotic cells. Examples of lentiviruses include human immunodeficiency virus (HIV) (e.g., strain 1 and strain 2), simian immunodeficiency virus (SIV), feline immunodeficiency virus (Hy), BLV, EIAV, CEV, and visna virus. Lentiviruses may be used for nondividing or terminally differentiated cells such as neurons, macrophages, hematopoietic stem cells, retinal photoreceptors, and muscle and liver cells, cell types for which previous gene therapy methods could not be used. A vector containing such a lentivirus core (e.g. gag gene) can transduce both dividing and non-dividing cells.

In certain embodiments, the viruses are adeno-associated viruses (AAVs). AAVs are naturally occurring defective viruses that require helper viruses to produce infectious particles (Muzyczka, N., Curr. Topics in Microbiol. Immunol. 158:97 (1992)). It is also one of the few viruses that can integrate its DNA into nondividing cells. Vectors containing as little as 300 base pairs of AAV can be packaged and can integrate, but space for exogenous DNA is limited to about 4.5 kb. In some cases, an AAV vector may include all the sequences necessary for DNA replication, encapsidation, and host-cell integration. The recombinant AAV vector can be transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Felgner, International Patent Publication Nos. WO 91/17424 and WO 91/16024. Delivery can be to cells (e.g. in vitro or ex vivo administration) or target tissues (e.g. in vivo administration). Physical methods of introducing polynucleotides may also be used. Examples of such methods include injection of a solution containing the polynucleotides, bombardment by particles covered by the polynucleotides, soaking a cell, tissue sample or organism in a solution of the polynucleotides, or electroporation of cell membranes in the presence of the polynucleotides.

Examples of delivery methods and vehicles include viruses, nanoparticles, exosomes, nanoclews, liposomes, lipids (e.g., LNPs), supercharged proteins, cell permeabilizing peptides, and implantable devices. The nucleic acids, proteins and other molecules, as well as cells described herein may be delivered to cells, tissues, organs, or subjects using methods described in paragraphs [00117] to [00278] of Feng Zhang et al., (International Patent Publication No. WO 2016/106236A1), which is incorporated by reference herein in its entirety.

In some cases, the methods include delivering the barcode construct and/or another element (e.g., a perturbation element) to cells. In such cases, the barcode construct and/or another element (e.g., a perturbation element) may be RNA molecules.

### Barcoded Libraries and Methods of Generating Thereof

The present disclosure further comprises barcoded libraries. The barcoded libraries may be generated by attaching (e.g., by trans-splicing) the barcoding constructs or portions thereof onto another nucleic acids. In some embodiments, the barcoded libraries comprise barcoding constructs attached with endogenous nucleic acids in cells. The endogenous nucleic acids may be genomic DNA, mitochondrial DNA, mRNA, rRNA, tRNA, exomal DNA, or any combination thereof. In some examples, the endogenous nucleic may be endogenous mRNA. The endogenous nucleic acids (e.g., the endogenous RNA molecules) in the barcoded library comprises one or more perturbations caused by the perturbation element.

The barcodes may be used for identifying the barcoded libraries. In some cases, members in the same barcoded library comprises a common barcode sequence that distinguish from members in other libraries. In some cases, in one or more cells expressing the same perturbation element, the members of the barcoded library comprise a common barcode sequence. In cases where the barcoded libraries comprising endogenous nucleic acids, the barcodes may be used for identifying cells or cell populations that contain the endogenous nucleic acids. For example, the endogenous nucleic acids in the same cell or cell population are attached with the same common barcode.

### Library Generation and Analysis

When the barcode sequences are spliced onto endogenous RNA molecules, nucleic acid libraries may be generated with the barcoded RNA molecules. In some cases, the barcoded RNA molecules may be isolated from cells (e.g., after lysing the cells) before the libraries are generated. In such cases, since the barcode sequences can be used to identify the perturbations and/or cell populations (e.g., cells of different lineages or different species), cells with different perturbations and/or of different population may be lysed in a single volume.

In general, the barcoded libraries may be isolated, reverse transcribed, and PCR amplified. In some embodiments, the generation of nucleic acid libraries include one or more of generating cDNA molecules from the barcoded RNA molecules by reverse transcription, and amplifying the cDNA molecules. The amplified cDNA molecules may be sequenced. In some cases, the amplified cDNA molecules may be fragmented and tagged (e.g., by fragmentation). The resulting nucleic acids may be further amplified (e.g., by step-in linear amplification) before sequencing. The barcoded libraries may be used for genome-wide expression profiling, e.g., performed using a combination of trans-splicing-specific primers and universal PCR primers, or two trans-splicing-specific primers may be employed in the amplification step. A universal primer flanking an amplification cassette may be introduced in the trans-spliced mRNA or cDNA using any suitable approach, including but not limited to, adaptor ligation, template-switching (e.g., using SMART™ technology by Clontech (Mountain View, Calif.) or ScriptSeg™ technology by Agilent (Santa Clara, Calif.)), tailing (e.g., using a terminal transferase), circularization (e.g., using CircLigase™ ssDNA ligase by Epicentre (Madison, Wis.)), linker ligation (e.g., using T4 RNA ligase), and/or any other suitable approach. According to one embodiment, the amplification primers incorporate specific sequences (e.g., adapter sequences) to facilitate a subsequent high-throughput (HT) sequencing step. In other aspects, the cDNA product generated after a reverse transcription step is amplified in a multiplex PCR assay (e.g., as described in the Experimental section herein). For example, the multiplex PCR may employ a mix of gene-specific primers and primer(s) specific for a trans-spliced mRNA or cDNA product. In certain aspects, the number of gene-specific PCR primers is 10 or more, 100 or more, 500 or more, or 1,000 or more, where each PCR primer is designed to target a specific sequence of one specific gene. Several multiplex primers may be designed for the same gene in order to profile different mRNA splice forms, or one primer may be designed for several distinct mRNAs to amplify mRNAs having related sequences. In certain aspects, the multiplex PCR primers include specific sequences (e.g. at the 5′-end) necessary for HT sequencing or multiplex HT sequencing.

### Elimination of Non-Spliced Constructs

In some embodiments, not all of the barcoding constructs are trans-spliced. Some barcoding constructs may be produced in cells but not trans-spliced. Such non-spliced barcoding constructs may contaminate the barcoded library generated later. Thus, the methods herein may further comprise eliminating non-spliced constructs. The elimination step may be performed after trans-splicing reactions occur and before sequencing. For example, the elimination step may be performed after an amplification step.

The elimination may be performed by specifically degrading or digesting the non-spliced constructs. In some embodiments, non-spliced barcoding constructs may be eliminated by a CRISPR-Cas system. Such CRISPR-Cas system may comprise guides that specifically recognizes (e.g., hybridize) to the trans-splicing element on the barcoding constructs (e.g., upstream of the splice acceptor site). If a trans-splicing reaction occurs, then the trans-splicing element is lost. If a trans-splicing reaction does not occur, then the trans-splicing element remains in cells and may be recognized by the guides. In such cases, the barcoding constructs comprising the trans-splicing elements may be removed by the nuclease in the CRISPR-Cas system.

In some embodiments, the elimination may be performed using affinity-based capture methods, e.g., hybrid capture. In some examples, the capture may be performed using beads. The beads may contain oligonucleotides that are complementary to the sequences upstream of the splice acceptor in the trans-splicing element. The beads may be magnetic. The molecules attached to the beads may be removed by magnetic separation or centrifugal separation.

In some embodiments, the elimination may be performed by enzyme digestion. Nucleases specifically recognizing the non-spliced constructs may be used. In some cases, the nucleases may be restriction endonucleases. In some cases, the polynucleotide herein may comprise one or more recognition sites of the nucleases.

### Amplification

The cDNA molecules generated from the barcoded library may be amplified. The amplification may be performed using unbiased amplification. Amplification may involve thermocycling or isothermal amplification (such as through the methods RPA or LAMP). For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is polymerase chain reaction (PCR). In particular, the isolated RNA can be subjected to a reverse transcription assay that is coupled with a quantitative polymerase chain reaction (RT-PCR) in order to quantify the expression level of a sequence associated with a signaling biochemical pathway.

### Sequencing

The methods herein may further include sequencing one or more members of the barcoded libraries or molecules derived therefrom. The sequence reads may be analyzed to determine the effects of perturbation on the mRNAs in cells, and the barcode sequence may be used to identify effects of a particular perturbation.

In some cases, the sequencing may be next generation sequencing. The terms “next-generation sequencing” or “high-throughput sequencing” refer to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies or single-molecule fluorescence-based method commercialized by Pacific Biosciences. Any method of sequencing known in the art can be used before and after isolation. In certain embodiments, a sequencing library is generated and sequenced.

At least a part of the processed nucleic acids and/or barcodes attached thereto may be sequenced to produce a plurality of sequence reads. The fragments may be sequenced using any convenient method. For example, the fragments may be sequenced using Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513:19-39) and Morozova et al (Genomics. 2008 92:255-64), which are incorporated by reference for the general descriptions of the methods and the particular steps of the methods, including all starting products, methods for library preparation, reagents, and final products for each of the steps. As would be apparent, forward and reverse sequencing primer sites that are compatible with a selected next generation sequencing platform can be added to the ends of the fragments during the amplification step. In certain embodiments, the fragments may be amplified using PCR primers that hybridize to the tags that have been added to the fragments, where the primer used for PCR have 5′ tails that are compatible with a particular sequencing platform. In certain cases, the primers used may contain a molecular barcode (an “index”) so that different pools can be pooled together before sequencing, and the sequence reads can be traced to a particular sample using the barcode sequence.

In some cases, the sequencing may be performed at certain “depth.” The terms “depth” or “coverage” as used herein refers to the number of times a nucleotide is read during the sequencing process. In regards to single cell RNA sequencing, “depth” or “coverage” as used herein refers to the number of mapped reads per cell. Depth in regards to genome sequencing may be calculated from the length of the original genome (G), the number of reads(N), and the average read length(L) as N×L/G. For example, a hypothetical genome with 2,000 base pairs reconstructed from 8 reads with an average length of 500 nucleotides will have 2× redundancy.

In some cases, the sequencing herein may be low-pass sequencing. The terms “low-pass sequencing” or “shallow sequencing” as used herein refers to a wide range of depths greater than or equal to 0.1× up to 1×. Shallow sequencing may also refer to about 5,000 reads per cell (e.g., 1,000 to 10,000 reads per cell).

In some cases, the sequencing herein may deep sequencing or ultra-deep sequencing. The term “deep sequencing” as used herein indicates that the total number of reads is many times larger than the length of the sequence under study. The term “deep” as used herein refers to a wide range of depths greater than 1× up to 100×. Deep sequencing may also refer to 100× coverage as compared to shallow sequencing (e.g., 100,000 to 1,000,000 reads per cell). The term “ultra-deep” as used herein refers to higher coverage (>100-fold), which allows for detection of sequence variants in mixed populations.

### Transcriptome Profiling

The methods herein may include determining the expression profile, e.g., the profile of a transcriptome. When a perturbation element is introduced or produced in a cell, the expression profile in the cell may be changed by the perturbation element. The expression profile may be analyzed to determine the effects of the perturbations.

According to certain embodiments, the expression profile includes “binary” or “qualitative” information regarding the expression of each gene of interest in a cell of interest. That is, in such embodiments, for each gene of interest, the expression profile only includes information that the gene is expressed or not expressed (e.g., above an established threshold level) in the target cell. In other embodiments, the expression profile includes quantitative information regarding the level of expression (e.g., based on rate of transcription, rate of splicing and/or RNA abundance) of one or more genes of interest. In certain aspects, the quantitative information regarding gene expression levels is obtained by measuring transcription and/or splicing (e.g., trans-splicing) of pre-mRNAs rather than the steady state levels of mature mRNAs, where the steady-state levels of mature mRNAs depends on additional processing, transport and turnover steps in the nucleus and cytoplasm.

According to one embodiment, when gene expression levels are based on transcription and/or splicing (e.g., trans-splicing) of pre-mRNAs, the transcribed and/or spliced pre-mRNAs measured are those present in the target cell within 12 hours, within 11 hours, within 10 hours, within 9 hours, within 8 hours, within 7 hours, within 6 hours, within 5 hours, within 4 hours, within 3 hours, within 2 hours, or within 1 hour or less after transduction of the target cell. In other aspects, gene expression levels are based on the steady state levels of mature mRNAs in the transduced target cell.

Expression profile may be detected using sequencing, e.g., high throughput sequencing as described herein. A single sequencing primer for sequencing the barcode element and gene-specific portion of the cDNA in a single read may be used. Alternatively, separate sequencing primers for the barcode element and gene-specific portion of the cDNA may be employed.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

Expression data may be generated using approaches other than HT sequencing. In certain aspects, quantitative RT-PCR (in single- or multi-plex) may be used to generate expression data, as described below in more detail. Other approaches for generating expression data may be employed, such as gene expression analysis using a hybridization assay (e.g. microarray technology (e.g., using a custom or pre-made microarray commercially available from Affymetrix, Agilent, or the like)) or nCounter® technology (NonoString Technologies, Seattle, Wash.), capillary electrophoresis-based methods, direct high-throughput sequencing of trans-spliced mRNAs or cDNAs (e.g. using HT sequencing technologies from Illumina, Inc. (San Diego, Calif.), Life Technologies (Carlsbad, Calif.), Pacific Biosciences (Menlo Park, Calif.), Helicos Biosciences (Cambridge, Mass.), etc.), or any other suitable approaches.

A qualitative and/or quantitative expression profile from the target cell may be compared to, e.g., a comparable expression profile generated from other target cells in the cellular sample and/or one or more reference profiles from cells known to have a particular biological phenotype or condition (e.g., a disease condition, such as a tumor cell; or treatment condition, such as a cell treated with an agent, e.g., a drug). When the profiles being compared are quantitative expression profiles, the comparison may include determining a fold-difference between one or more genes in the expression profile of a target cell and the corresponding genes in the expression profile(s) of one or more different target cells in the cellular sample, or the corresponding genes in a reference cell or cellular sample. Alternatively, or additionally, the single cell expression profile may include information regarding the relative expression levels of different genes in a single target cell. In certain aspects, the fold difference in intercellular expression levels or intracellular expression levels can be determined to be 0.1 or more, 0.5 fold or more, 1 fold or more, 1.5 fold or more, 2 fold or more, 2.5 fold or more, 3 fold or more, 4 fold or more, 5 fold or more, 6 fold or more, 7 fold or more, 8 fold or more, 9 fold or more, or more than 10 fold or more, for example.

The expression profile may be indicative of the biological condition of the cell including, but not limited to, a disease condition (e.g., a cancerous condition, metastatic potential, an epithelial mesenchymal transition (EMT) characteristic, and/or any other disease condition of interest), the condition of the cell in response to treatment with any physical action (e.g., heat shock, hypoxia, normoxia, hydrodynamic stress, radiation, and/or the like), the condition of the cell in response to treatment with chemical compounds (e.g., drugs, cytotoxic agents, nutrients, salts, and/or the like) or biological extracts or entities (e.g., viruses, bacteria, other cell types, growth factors, biologics, and/or the like), and/or any other biological condition of interest (e.g. immune response, senescence, inflammation, motility, and/or the like). The expression profile may be used to reveal heterogeneity in the target cell population and classify (or sub-classify) a target cell within a cellular sample (e.g., a clinical sample).

### Whole-Organism Barcoding

The methods and compositions herein may also be used for whole-organism RNA barcoding, where RNA can be retrieved from an entire organism and mapped to a particular cell type, tissue, organ, or lineage. In some examples, a transgenic organism can be generated. The organism may have one or more barcodes expressed via one or more cell-specific, tissue-specific or organ-specific promoters or enhancers. In some cases, the linkage or mapping between barcodes and promoters is known, thus the barcodes may be used to measure RNA in cells, tissues or organs of interest. With the methods described herein, one can harvest bulk RNA samples from this transgenic organism and then use the barcodes to measure RNA in the cells, tissues, or organs of interest.

In some examples, a method of performing whole-organism barcoding in a subject, comprising delivering a plurality of polynucleotides into multiple types of cells in the subject, each polynucleotide comprising a sequence encoding a barcoding construct operably linked to an antisense promoter, wherein the barcoding construct comprises a trans-splicing element and a barcode sequence, and the antisense promoter is a cell-specific promoter; in each cell, generating RNA transcripts of the polynucleotides, wherein the transcripts comprise the barcoding constructs; and splicing each of the barcoding sequence onto endogenous RNA molecules in the cells, wherein cells in the same type of cells comprise a common barcode sequence and the barcode sequence in each type of cells is unique. The subject may be a genetically modified organism (e.g., a transgenic organism).

### KITS

Further provided herein include kits for performing the methods herein. A kit may comprise one or more of the nucleic acids such as the polynucleotides, barcoding constructs, perturbation elements described herein. The kit may also comprise cells, viruses, and reagents needed for performing the methods.

In addition to reagents and devices, the kits may further include instructions for using the components of the kit to practice the methods. The instructions for practicing the subject methods may be generally recorded on a suitable recording medium. For example, the instructions may be printed on a substrate, such as paper or plastic, etc. As such, the instructions may be present in the kits as a package insert, in the labeling of the container of the kit or components thereof. In other embodiments, the instructions are present as an electronic storage data file present on a suitable computer readable storage medium, e.g., CD-ROM, diskette, etc. In certain embodiments, the instructions are not present in the kit, but means for obtaining the instructions from a remote source, e.g., via the internet, are provided.

The present application also provides aspects and embodiments as set forth in the following numbered Statements:

Statement 1. A nucleic acid construct comprising: a nucleic acid sequence encoding i) a barcoding construct operably linked to a first promoter that is an antisense promoter and comprises a trans-splicing element and a barcode sequence, and a nucleic acid sequence encoding one or more perturbation elements operably linked to a second promoter.

Statement 2. The nucleic acid construct of Statement 1, further comprising a nucleic acid sequence encoding a transcription terminator.

Statement 3. The nucleic acid construct of any one of the proceeding Statements, wherein the transcription terminator is an antisense terminator.

Statement 4. The nucleic acid construct of any one of the proceeding Statements, wherein the antisense promoter does not comprise a splice donor site.

Statement 5. The nucleic acid construct of any one of the proceeding Statements, further comprising a reverse transcription primer binding site.

Statement 6. The nucleic acid construct of any one of the proceeding Statements, wherein the trans-splicing element comprises: a branch point, a polypyrimidine tract, a splice acceptor sequence, or a combination thereof.

Statement 7. The nucleic acid construct of any one of the proceeding Statements, wherein the trans-splicing element is a ribozyme.

Statement 8. The nucleic acid construct of any one of the proceeding Statements, further comprising a CRISPR-Cas guide RNA binding site.

Statement 9. The nucleic acid construct of any one of the proceeding Statements, wherein the CRISPR-Cas guide RNA binding site is upstream of a transcribed trans-splicing element.

Statement 10. The nucleic acid construct of any one of the proceeding Statements, wherein the one or more perturbation elements comprises ORF sequences, guide RNAs, siRNAs, shRNAs, miRNAs, tRNAs, snRNAs, or lncRNAs.

Statement 11. The nucleic acid construct of any one of the proceeding Statements, wherein the one or more perturbation elements comprises an snRNA.

Statement 12 The nucleic acid construct of any one of the proceeding Statements, wherein the one or more perturbation elements comprises a guide RNA.

Statement 13. The nucleic acid construct of any one of the proceeding Statements, wherein the antisense promoter is a cell-specific, tissue-specific, or organ-specific promoter.

Statement 14. A vector comprising the nucleic acid construct of any one of the preceding Statements.

Statement 15. The vector of Statement 14, wherein the vector is a viral vector.

Statement 16. The vector of Statement 14 or 15, wherein the viral vector is a lentiviral vector.

Statement 17. A method of generating a barcoded nucleic acid library, comprising: delivering one or more polynucleotides into a cell, each polynucleotide comprising: a sequence encoding a barcoding construct operably linked to a first promoter that is an antisense promoter, wherein the barcoding construct comprises a trans-splicing element and a barcode sequence; and a sequence encoding a perturbation element operably linked to a second promoter; generating RNA transcripts of the one or more polynucleotide delivered into the cell, wherein the RNA transcripts comprise the barcoding construct and the perturbation element; and splicing the barcoding sequence onto endogenous RNA molecules in the cell, thereby generating a barcoded library, each member of the barcoded library comprising the barcode sequence and the endogenous RNA molecules attached with the barcode sequence.

Statement 18. The method of Statement 17, wherein each member of the barcoded library comprises a common barcode sequence.

Statement 19. The method of Statement 17 or 18, further comprising delivering a plurality of polynucleotides to a plurality of cells, wherein the members of the barcoded library generated in each cell comprise a unique barcode.

Statement 20. The method of any one of Statements 17-19, wherein the plurality of polynucleotides comprises sequences encoding at least 1000 perturbation elements.

Statement 21. The method of any one of Statements 17-20, wherein the plurality of cells comprise a plurality of barcoded libraries, and the method further comprises lysing the plurality of cells in a single volume.

Statement 22. The method of any one of Statements 17-21, wherein the one or more polynucleotides is in a viral vector.

Statement 23. The method of any one of Statements 17-22, wherein the viral vector is a lentiviral vector.

Statement 24. The method of any one of Statements 17-23, wherein a strength of the first promoter is weaker than a strength of the second promoter.

Statement 25. The method of any one of Statements 17-24, wherein the first promoter does not comprise a splice donor site.

Statement 26. The method of any one of Statements 17-25, wherein the one or more polynucleotides further comprises a sequence encoding a transcription terminator.

Statement 27. The method of any one of Statements 17-26, wherein the transcription terminator is an antisense sequence.

Statement 28. The method of any one of Statements 17-27, further comprising eliminating non-spliced barcoding constructs.

Statement 29. The method of any one of Statements 17-28, wherein the non-spliced barcoding constructs are eliminated by a CRISPR-Cas system.

Statement 30. The method of any one of Statements 17-29, further comprising sequencing the barcode sequence and the endogenous RNA molecules.

Statement 31. The method of any one of Statements 17-30, wherein one or more of the endogenous RNA molecules in the barcoded library comprises a perturbation caused by the perturbation element.

Statement 32. The method of any one of Statements 17-31, wherein the polynucleotide is delivered by virus transduction.

Statement 33. The method of any one of Statements 17-32, wherein the perturbation element comprise ORF sequences, mRNAs, guide RNAs, siRNAs, shRNAs, miRNAs, tRNAs, rRNAs, snRNAs, or lncRNAs.

Statement 34. The method of any one of Statements 17-33, wherein the barcoding construct further comprises a reverse transcription primer binding site.

Statement 35. The method of any one of Statements 17-34, wherein the trans-splicing element comprises a branch point, a polypyrimidine tract, a splice acceptor sequence, or a combination thereof.

Statement 36. The method of any one of Statements 17-35, wherein the trans-splicing element is a ribozyme.

Statement 37. The method of any one of Statements 17-36, wherein the ribozyme comprises Tetrahymena group I intron or Azoarcus group I intron.

Statement 38. The method of any one of Statements 17-37, wherein the first or the second prompter is a SV40, CMV, U6, or EF1a promoter.

Statement 39. The method of any one of Statements 17-38, further comprising generating cDNA molecules from the barcoded library.

Statement 40. The method of any one of Statements 17-39, wherein the barcode sequence is flanked by at least one filter sequence.

Statement 41. The method of any one of Statements 17-40, further comprising sequencing at least a portion of the barcode sequence and at least a portion of endogenous RNA molecule attached thereto.

Statement 42. The method of any one of Statements 17-41, further comprising amplifying the barcoded library.

Statement 43. The method of any one of Statements 17-42, wherein the amplification is unbiased amplification.

Statement 44. The method of any one of Statements 17-43, wherein the endogenous RNA is mRNA.

Statement 45. The method of any one of Statements 17-44, wherein the first promoter is a cell-specific, tissue-specific, or organ-specific promoter.

Statement 46. A method of labeling cell populations, comprising: delivering a plurality of polynucleotides into a plurality of cell populations, each polynucleotide comprising a sequence encoding a barcoding construct operably linked to an antisense promoter, wherein the barcoding construct comprises a trans-splicing element and a barcode sequence; in each cell, generating RNA transcripts of the polynucleotides, wherein the transcripts comprise the barcoding constructs; splicing each of the barcoding sequence onto endogenous RNA molecules in the cells, wherein cells in the same cell population comprise a common barcode sequence and the barcode sequence in each cell population is unique.

Statement 47. The method of Statement 46, wherein cells in each population are of the same lineage.

Statement 48. The method of any one of Statements 46-47, wherein cells in each population are from or derived from the same species.

Statement 49. A method of performing whole-organism barcoding in a subject, comprising: delivering a plurality of polynucleotides into multiple types of cells in the subject, each polynucleotide comprising a sequence encoding a barcoding construct operably linked to an antisense promoter, wherein the barcoding construct comprises a trans-splicing element and a barcode sequence, and the antisense promoter is a cell-specific promoter; in each cell, generating RNA transcripts of the polynucleotides, wherein the transcripts comprise the barcoding constructs; and splicing each of the barcoding sequence onto endogenous RNA molecules in the cells, wherein cells in the same type of cells comprise a common barcode sequence and the barcode sequence in each type of cells is unique.

Statement 50. The method of Statement 49, wherein the subject is a transgenic organism.

Statement 51. The method of Statement 49 or 50, further comprising sequencing the barcode sequence and the endogenous RNA.

The invention is further described in the following examples, which do not limit the scope of the invention described in the claims.

### EXAMPLES

### Example 1—Trans-Splicing Transcriptome Barcoding for Lineages and Perturbations

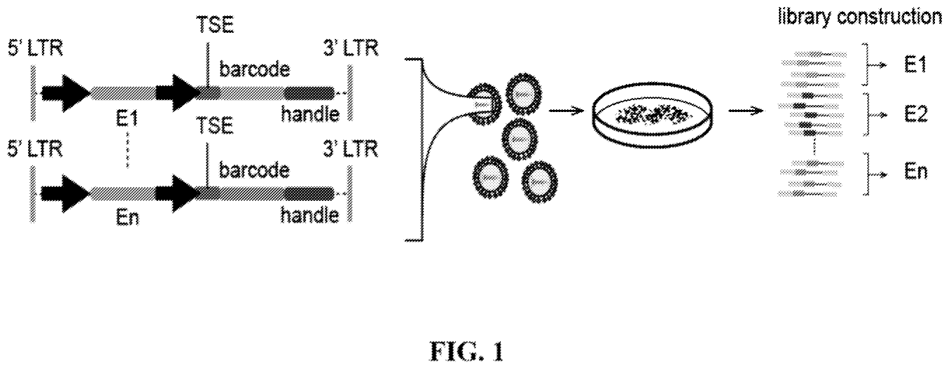

Lentivirus constructs such as the one shown in FIG. 1 were used for trans-splicing based transcriptome barcoding. In this particular example, elements (E1 through En) from a perturbation library (such as ORFs, mRNAs, sgRNAs, siRNAs, shRNAs, miRNAs, tRNAs, rRNAs, snRNAs or lncRNAs) have a cognate nucleic acid barcode (shown in color), driven by a separate promoter (such as CMV or SV40). In the context of lineage barcoding, a single-promoter system driving the barcoding construct was used. The barcoding construct was comprised of a 1) promoter ii) trans-splicing element (such as ribozyme, or a spliceosome splice-acceptor) iii) a nucleic acid barcode iv) a reverse-transcription handle and v) transcription termination sequence. Two examples of a trans-splicing elements (TSE) were used a 1) a spliceosome-mediated trans-splicing element comprising branch point (BP) and polypyrimidine tract (PPT) followed by a splice acceptor sequence (such as YAGG) and ii) a trans-splicing ribozyme, such as the Tetrahymena group I intron or Azoarcus group I intron ribozymes. Such ribozymes allow for a barcode and reverse-transcription handle to be ligated to endogenous transcripts via trans-splicing.

Overall the trans-splicing barcoding approach allows for one-pot RNAseq library construction from cells with a library of perturbations (or several lineages). Thus, complex libraries or mixtures of lineages can be lysed in one single tube and the RNAseq information from each perturbation (or lineage) can be subsequently mapped via sequencing of the nucleic acid barcodes without the need for droplet-based or hydrogel-based compartmentalization.

Using paired-end next-generation sequencing (NGS), a sequencing read can provide both 1) the nucleic acid barcode (thus the perturbation or lineage information) and ii) the cDNA sequence to allow for transcriptome reconstruction. In some cases, the nucleic acid barcode can be flanked by two known filter sequences in order to confidently identify the nucleic acid barcode in the NGS read.

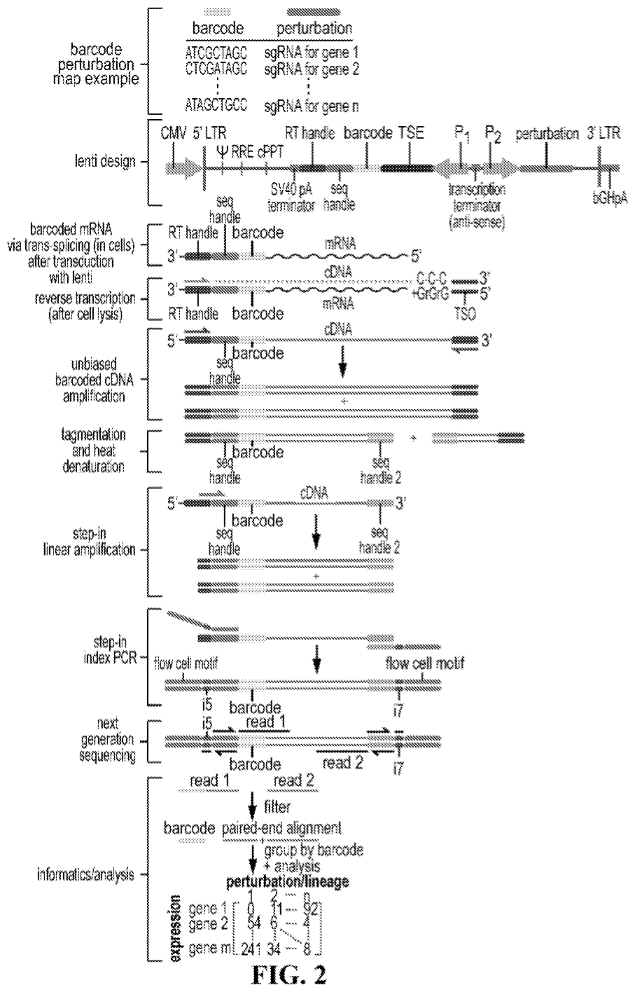

FIG. 2 shows a flowchart outlining the method for generating barcoded libraries. Optionally after the step-in linear amplification, Cas9 based elimination of non-trans-spliced TSEs during library construction may be performed.

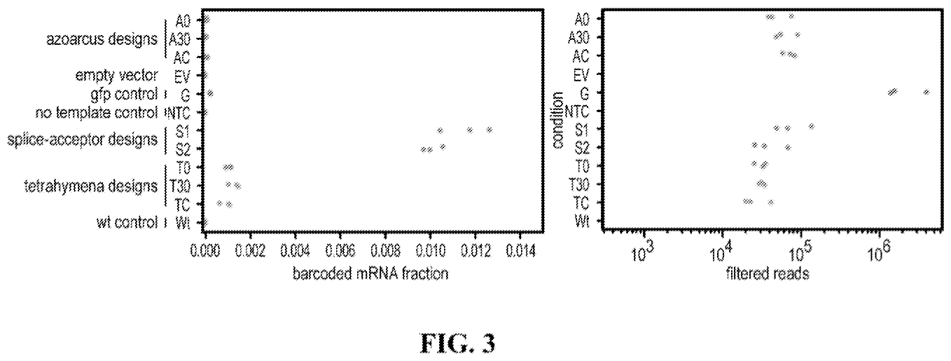

293T cell lines were made using lentivirus with several vectors. Using an SV40 promoter, Applicants show two classes of barcodes that generate barcoded mRNAs via trans-splicing: i) spliceosome-mediated trans-splicing elements and ii) group I intron ribozymes. The results shown in FIG. 3.

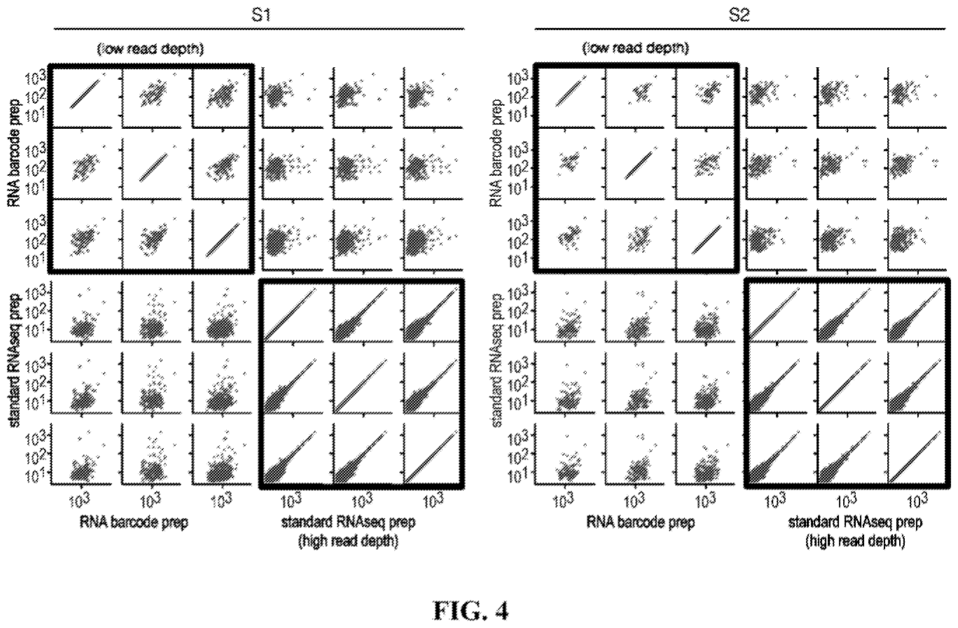

Based on shallow sequencing of trans-splicing elements S1 and S2 in FIG. 3, trans-spliced reads showed quantitative nature, as shown by top left quadrant of each RNAseq plot. Standard RNAseq preps had deeper sequencing, thus showing more genes and higher correlation. The results are shown in FIG. 4.

Further, Applicants tested barcoding cells with different species origins. 293T (human) and 3T3 (mouse) cell lines were labeled with two different nucleic acid barcodes, using the S1 construct (spliceosome-mediated RNA barcoding) to test whether trans-splicing based transcriptome barcoding was indeed specific and that transcriptomes could indeed be reconstructed from pooled lysis and sequencing. Results show that human and mouse transcripts were both detected in the pool (293T cells co-cultured with 3T3 cells). However, when the barcodes were used to label reads, reads labeled with barcode A mapped to the human transcriptome, whereas reads labeled with barcode B mapped to the mouse transcriptome. The results demonstrate that trans-splicing based transcriptome barcoding was indeed specific (barcode A maps to human, barcode B maps to mouse) and that barcoding events happened within the cells.

### Example 2—RNA Barcoding

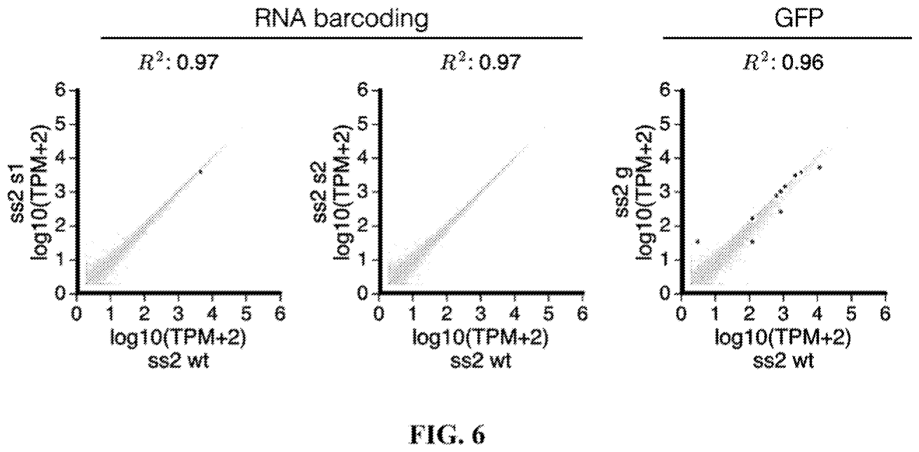

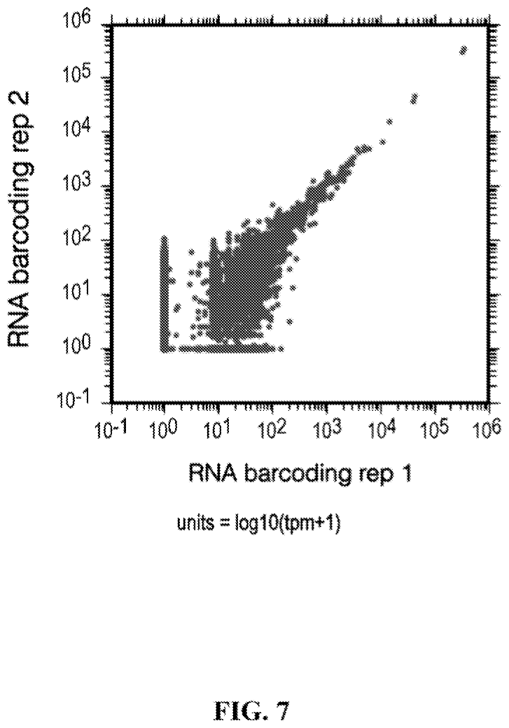

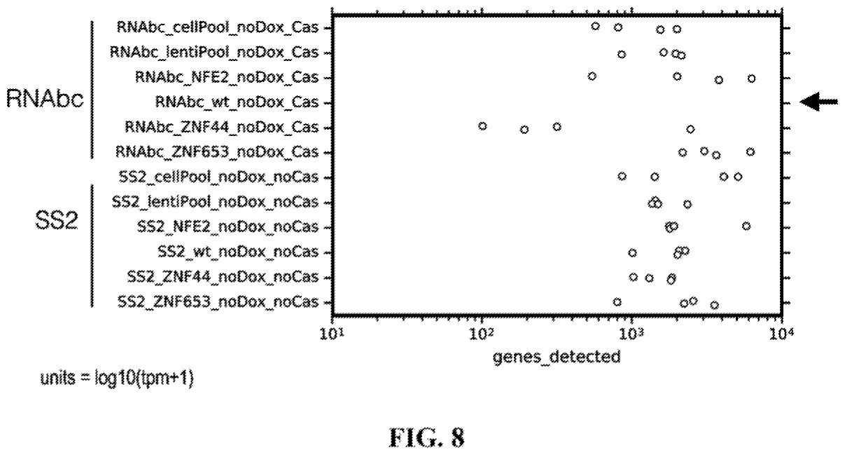

RNA barcoding using the methods herein were tested. RNAseq was conducted on 293T cells expressing RNA barcoding constructs, showing no differentially expressed genes (FIG. 6). The results show that the RNA barcoding was not perturbative. Further, FIG. 7 shows that the RNA barcoding was quantitative. Two RNA barcoding biological replicates showed high correlation and quantitative behavior via RNAseq. RNAseq with RNA barcoding (RNAbc) showed comparable genes detected to state-of-the-art SMART-SEQ2 (SS2), demonstrating high information content. The negative control (arrow) showed that wild-type 293T cells did not produce any barcoded reads when performing the RNA barcode library construction (FIG. 8).

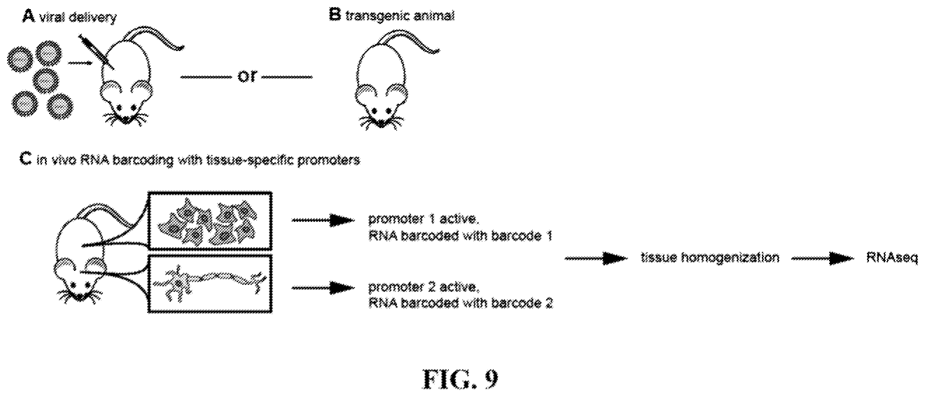

The RNA barcoding approach may also be used in vivo. FIG. 9 shows an exemplary method of whole-organism barcoding. By using cell-specific, tissue-specific, or organ-specific promoters, one can deliver a library of barcodes (A) or make a transgenic animal with a library of barcodes (B) to barcode RNA in vivo. C) In vivo RNA barcoding allows for RNAseq to be carried out on desired cell populations without having to do flow-cytometry and/or single-cell sequencing.

### Example 3—RNA Barcoding with ORF Library

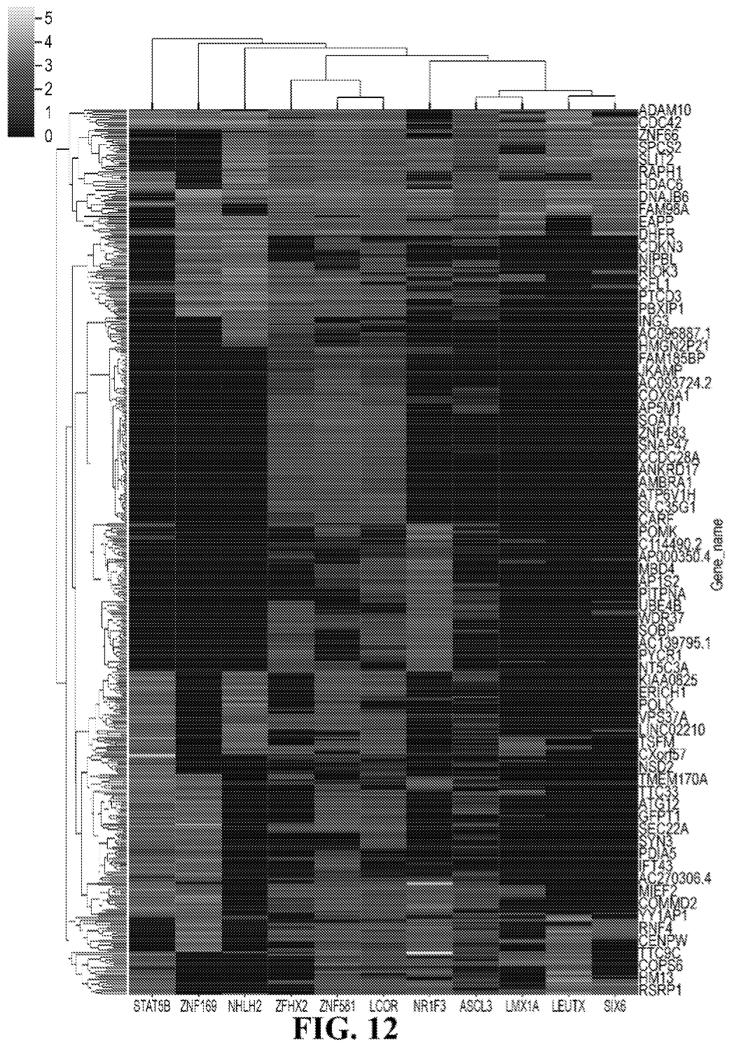

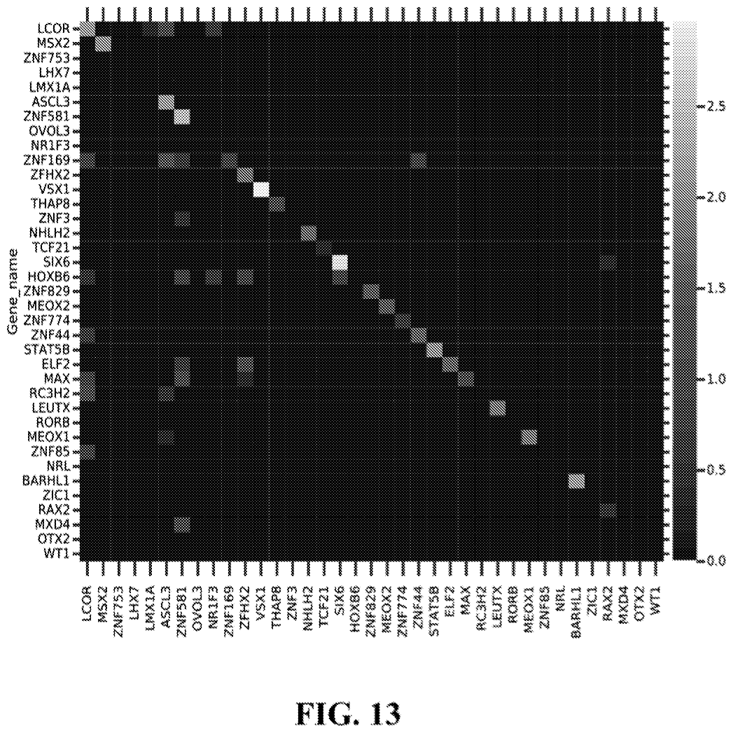

An ORF library was cloned into a lentivirus vector with a cognate trans-splicing RNA barcode. Using lentivirus generated from these constructs, HEK293FT cells were stably transduced to express the ORF and trans-splicing RNA barcodes. Each ORF was paired with a unique barcode, and transcriptomes were successfully reconstructed for each ORF perturbation. Expression of transcripts is denoted in log 10 scale transformed transcripts per million (TPM). FIG. 12 shows the transcriptomes of a cell library of 11 pooled ORFs with unique barcodes. FIG. 13 shows the expression levels of the ORF library. Most ORFs were barcoded by their corresponding trans-splicing barcode.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A nucleic acid construct, comprising:
a nucleic acid sequence encoding i) a barcoding construct operably linked to a first promoter that is an antisense promoter and comprises a trans-splicing element and a barcode sequence; and
a nucleic acid sequence encoding one or more perturbation elements operably linked to a second promoter.

2. The nucleic acid construct of claim 1, further comprising a nucleic acid sequence encoding a transcription terminator.

3. The nucleic acid construct of claim 2, wherein the transcription terminator is an antisense terminator.

4. The nucleic acid construct of claim 1, wherein the antisense promoter does not comprise a splice donor site.

5. The nucleic acid construct of claim 1, further comprising a reverse transcription primer binding site.

6. The nucleic acid construct of claim 1, wherein the trans-splicing element comprises:
a. a branch point;
b. a polypyrimidine tract;
c. a splice acceptor sequence; or
d. a combination thereof.

7. The nucleic acid construct of claim 1, wherein the trans-splicing element is a ribozyme.

8. The nucleic acid construct of claim 1, further comprising a CRISPR-Cas guide RNA binding site.

9. The nucleic acid construct of claim 8, wherein the CRISPR-Cas guide RNA binding site is upstream of a transcribed trans-splicing element.

10. The nucleic acid construct of claim 1, wherein the one or more perturbation elements comprises ORF sequences, guide RNAs, siRNAs, shRNAs, miRNAs, tRNAs, snRNAs, or lncRNAs.

11. The nucleic acid construct of claim 1, wherein the one or more perturbation elements comprises an snRNA.

12. The nucleic acid construct of claim 1, wherein the one or more perturbation elements comprises a guide RNA.

13. The nucleic acid construct of claim 1, wherein the antisense promoter is a cell-specific, tissue-specific, or organ-specific promoter.

14. A vector comprising the nucleic acid construct of any one of the preceding claims.

15. The vector of claim 14, wherein the vector is a viral vector.

16. The vector of claim 15, wherein the viral vector is a lentiviral vector.

17. A method of generating a barcoded nucleic acid library, comprising:
a. delivering one or more polynucleotides into a cell, each polynucleotide comprising:
i. a sequence encoding a barcoding construct operably linked to a first promoter that is an antisense promoter, wherein the barcoding construct comprises a trans-splicing element and a barcode sequence; and
ii. a sequence encoding a perturbation element operably linked to a second promoter;
b. generating RNA transcripts of the one or more polynucleotides delivered into the cell, wherein the RNA transcripts comprise the barcoding construct and the perturbation element; and
c. splicing the barcoding sequence onto endogenous RNA molecules in the cell, thereby generating a barcoded library, each member of the barcoded library comprising the barcode sequence and the endogenous RNA molecules attached with the barcode sequence.

18. The method of claim 17, wherein each member of the barcoded library comprises a common barcode sequence.

19. The method of claim 17, further comprising delivering a plurality of polynucleotides to a plurality of cells, wherein the members of the barcoded library generated in each cell comprise a unique barcode.

20. The method of claim 19, wherein the plurality of polynucleotides comprises sequences encoding at least 1,000 perturbation elements.

21. The method of claim 19, wherein the plurality of cells comprise a plurality of barcoded libraries, and the method further comprises lysing the plurality of cells in a single volume.

22. The method of claim 17, wherein the one or more polynucleotides is in a viral vector.

23. The method of claim 22, wherein the viral vector is a lentiviral vector.

24. The method of claim 1, wherein a strength of the first promoter is weaker than a strength of the second promoter.

25. The method of claim 1, wherein the first promoter does not comprise a splice donor site.

26. The method of claim 1, wherein the one or more polynucleotides further comprise a sequence encoding a transcription terminator.

27. The method of claim 26, wherein the transcription terminator is an antisense sequence.

28. The method of claim 17, further comprising eliminating non-spliced barcoding constructs.

29. The method of claim 28, wherein the non-spliced barcoding constructs are eliminated by a CRISPR-Cas system.

30. The method of claim 17, further comprising sequencing the barcode sequence and the endogenous RNA molecules.

31. The method of claim 17, wherein one or more of the endogenous RNA molecules in the barcoded library comprises a perturbation caused by the perturbation element.

32. The method of claim 17, wherein the one or more polynucleotides is delivered by virus transduction.

33. The method of claim 17, wherein the perturbation element comprises ORF sequences, mRNAs, sgRNAs, siRNAs, shRNAs, miRNAs, tRNAs, rRNAs, snRNAs, or lncRNAs.

34. The method of claim 17, wherein the barcoding construct further comprises a reverse transcription primer binding site.

35. The method of claim 17, wherein the trans-splicing element comprises:
a. a branch point;
b. a polypyrimidine tract;
c. a splice acceptor sequence; or
d. a combination thereof.

36. The method of claim 17, wherein the trans-splicing element is a ribozyme.

37. The method of claim 36, wherein the ribozyme comprises Tetrahymena group I intron or Azoarcus group I intron.

38. The method of claim 17, wherein the first or the second prompter is a SV40, CMV, U6, or EF1a promoter.

39. The method of claim 17, further comprising generating cDNA molecules from the barcoded library.

40. The method of claim 17, wherein the barcode sequence is flanked by at least one filter sequence.

41. The method of claim 17, further comprising sequencing at least a portion of the barcode sequence and at least a portion of the endogenous RNA molecules attached thereto.

42. The method of claim 17, further comprising amplifying the barcoded library.

43. The method of claim 42, wherein the amplification is unbiased amplification.

44. The method of claim 17, wherein the endogenous RNA molecules are mRNA.

45. The method of claim 17, wherein the first promoter is a cell-specific, tissue-specific, or organ-specific promoter.

46. A method of labeling cell populations, comprising:
a. delivering a plurality of polynucleotides into a plurality of cell populations, each polynucleotide comprising a sequence encoding a barcoding construct operably linked to an antisense promoter, wherein the barcoding construct comprises a trans-splicing element and a barcode sequence;
b. in each cell, generating RNA transcripts of the polynucleotides, wherein the transcripts comprise the barcoding constructs; and
c. splicing each of the barcoding sequence onto endogenous RNA molecules in the cells, wherein cells in the same cell population comprise a common barcode sequence and the barcode sequence in each cell population is unique.

47. The method of claim 46, wherein cells in each population are of the same lineage.

48. The method of claim 46, wherein cells in each population are from or derived from the same species.

49. A method of performing whole-organism barcoding in a subject, comprising:
a. delivering a plurality of polynucleotides into multiple types of cells in the subject, each polynucleotide comprising a sequence encoding a barcoding construct operably linked to an antisense promoter, wherein the barcoding construct comprises a trans-splicing element and a barcode sequence, and the antisense promoter is a cell-specific promoter;
b. in each cell, generating RNA transcripts of the polynucleotides, wherein the transcripts comprise the barcoding constructs; and
c. splicing each of the barcoding sequences onto endogenous RNA molecules in the cells, wherein cells in the same type of cells comprise a common barcode sequence and the barcode sequence in each type of cells is unique.

50. The method of claim 49, wherein the subject is a transgenic organism.

51. The method of claim 49, further comprising sequencing the barcode sequence and the endogenous RNA molecules.
